(19) United States
(12) Patent Application Publication
(10) Pub. No.: US 2008/0249113 A1
(43) Pub. Date: Oct. 9, 2008

(54) Fungicidal Mixtures

(76) Inventors: Carsten Blettner, Hong Kong (CN); Jochen Dietz, Mannheim (DE); Wassilios Grammenos, Ludwigshafen (DE); Thomas Grote, Wachenheim (DE); Udo Hunger, Mainz (DE); Bernd Muller, Frankenthal (DE); Matthias Niedenbruck, Limburgerhof (DE); Joachim Rheinheimer, Ludwigshafen (DE); Peter Schafer, Ottersheim (DE); Frank Schieweck, Hessheim (DE); Anja Schwogler, Mannheim (DE); Barbara Nave, Deidesheim (DE); Maria Scherer, Landau (DE); Siegfried Strathmann, Limburgerhof (DE); Ulrich Schofl, Bruhl (DE); Reinhard Stierl, Freinsheim (DE)

Correspondence Address: BIRCH STEWART KOLASCH & BIRCH, PO BOX 747, FALLS CHURCH, VA, 22040-0747, US

(21) Appl. No.: 11/659,192

(22) PCT Filed: Jul. 28, 2005
(86) PCT No.: PCT/EP05/08192

§ 371 Date: Feb. 2, 2007

(30) Foreign Application Priority Data
Aug. 3, 2004 (DE) 10 2004 037 784.7

(51) Int. Cl. A01N 43/90; C07D 487/04; A01N 25/26; A01P 3/00

(52) U.S. Cl. 514/259.31; 544/263; 504/100

(57) ABSTRACT

Fungicidal mixtures, comprising as active components
1) a 5-chloro-6-phenyl-7-heterocyclylaminotriazolopyrimidine of the formula I,

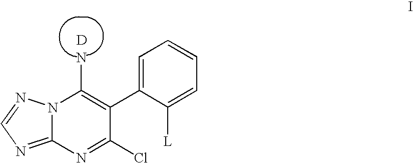

in which
D together with the nitrogen atom forms a pyrrolidine, piperidine or azepine ring, which rings are unsubstituted or substituted by one or two methyl groups or by an ethyl, propyl or butyl group; and
L is methyl, fluorine or chlorine;
and
2) at least one active compound II selected from the following groups:
   A) azoles;
   B) strobilurins;
   C) acylalanines;
   D) amine derivatives;
   E) anilinopyrimidines;
   F) dicarboximides;
   G) cinnamides and analogs;
   H) antibiotics;
   K) dithiocarbamates;
   L) heterocyclic compounds according to the description;
   M) sulfur and copper fungicides;
   N) nitrophenyl derivatives;
   O) phenylpyrroles;
   P) sulfenic acid derivatives;
   Q) other fungicides according to the description; or
   R) growth retardants;
      in a synergistically effective amount, novel triazolopyrimidines, methods for controlling harmful fungi using compounds or mixtures of a compound I with an active compound of groups A) to R) and the use of the compounds I with the active compounds of groups A) to R) for preparing such mixtures, and also compositions comprising these compounds or mixtures.

The present invention relates to fungicidal mixtures, comprising as active components

1) a 5-chloro-6-phenyl-7-heterocyclylaminotriazolopyrimidine of the formula I,

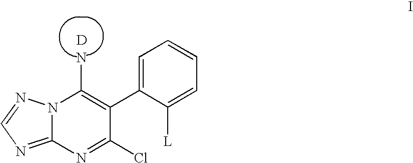

in which
D together with the nitrogen atom forms a pyrrolidine, piperidine or azepine ring, which rings are unsubstituted or substituted by one or two methyl groups or by an ethyl, propyl or butyl group; and
L is methyl, fluorine or chlorine;
   and

2) at least one active compound II selected from the following groups:
A) azoles, such as bitertanol, bromoconazole, cyproconazole, difenoconazole, dinitroconazole, epoxiconazole, fenbuconazole, fluquinconazole, flusilazole, flutriafol, hexaconazole, imazalil, ipconazole, metconazole, myclobutanil, penconazole, propiconazole, prochloraz, prothioconazole, simeconazole, tebuconazole, tetraconazole, triadimefon, triadimenol, triflumizole, triticonazole or methyl 2-(ortho-(2,5-dimethylphenyloxy-methylene)phenyl)-3-methoxyacrylate;
B) strobilurins, such as azoxystrobin, dimoxystrobin, fluoxastrobin, kresoxim-methyl, metominostrobin, orysastrobin, picoxystrobin, pyraclostrobin, trifloxystrobin or methyl 2-(ortho-(2,5-dimethylphenyloxymethylene)phenyl)-3-methoxyacrylate;
C) acylalanines, such as benalaxyl, metalaxyl, mefenoxam, ofurace, oxadixyl;
D) amine derivatives, such as aldimorph, dodine, dodemorph, fenpropimorph, fenpropidin, guazatine, iminoctadine, spiroxamine, tridemorph;
F) dicarboximides, such as iprodione, myclozolin, procymidone, vinclozolin;
G) cinnamides and analogs, such as dimethomorph, flumetover or flumorph;
H) antibiotics, such as cycloheximide, griseofulvin, kasugamycin, natamycin, polyoxin or streptomycin;
K) dithiocarbamates, such as ferbam, nabam, maneb, mancozeb, metam, metiram, propineb, polycarbamate, thiram, ziram, zineb;
L) heterocyclic compounds, selected from anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolan, mepronil, nuarimol, picobenzamid, probenazole, proquinazid, pyrifenox, pyroquilon, quinoxyfen, silthiofam, thiabendazole, thifluzamide, thiophanate-methyl, tiadinil, tricyclazole, triforine, 3-[5-(4-chloro-phenyl)-2,3-dimethylisoxazolidin-3-yl]pyridine, methyl (2-chloro-5-[1-(3-methylbenzyloxyimino)ethyl]benzyl)carbamate, methyl (2-chloro-5-[1-(6-methylpyridin-2-yl-methoxyimino)ethyl]benzyl)carbamate,
   benzimidazole derivatives of the formula IIA

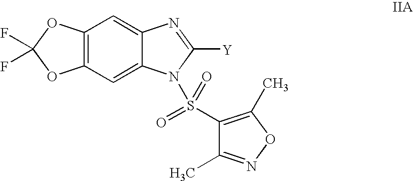

in which Y is chlorine or bromine;
sulfamoyl compounds of the formula III

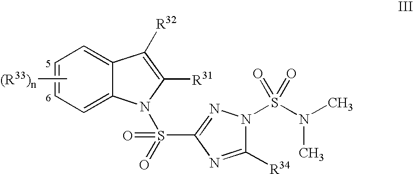

in which the substituents are as defined below:
R³¹ is hydrogen, halogen, cyano, C₁-C₄-alkyl, C₁-C₂-haloalkyl, C₁-C₄-alkoxy, C₁-C₄-alkylthio, C₁-C₄-alkoxycarbonyl, phenyl, benzyl, formyl or CH═NOR³¹¹;
   R³¹¹ is hydrogen, C₁-C₄-alkyl, C₁-C₄-alkylcarbonyl;
R³² is hydrogen, halogen, cyano, C₁-C₄-alkyl, C₁-C₂-haloalkyl, C₁-C₆-al-koxycarbonyl;
R³³ is halogen, cyano, nitro, C₁-C₄-alkyl, C₁-C₂-haloalkyl, C₁-C₄-alkoxy, C₁-C₄-alkylthio, C₁-C₄-alkoxycarbonyl, formyl or CH═NOR³¹¹;
n is 0, 1, 2, 3 or 4;
R³⁴ is hydrogen, halogen, cyano, C₁-C₄-alkyl or C₁-C₂-haloalkyl;
or
thiophene derivatives of the formula IV

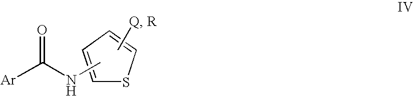

in which the variables are as defined below:
Ar is phenyl or a five- or six-membered aromatic heterocycle which contains one to four heteroatoms from the group consisting of O, N and S, where the cycles are unsubstituted or may be substituted by one to three groups R⁴¹:
R⁴¹ is halogen, C₁-C₄-alkyl or C₁-C₄-haloalkyl;
R is phenyl, C₁-C₈-alkyl, C₁-C₈-haloalkyl, C₁-C₈-alkoxy, C₁-C₈-halo-alkoxy;
Q is hydrogen, C₁-C₈-alkyl, C₁-C₈-haloalkyl, C₁-C₈-alkoxy, C₁-C₈-haloalkoxy;
M) sulfur and copper fungicides, such as Bordeaux mixtures, copper acetate, copper oxychloride, basic copper sulfate;
N) nitrophenyl derivatives, such as binapacryl, dinocap, dinobuton, nitrophthal-isopropyl;
O) phenylpyrroles, such as fenpiclonil or fludioxonil;
P) sulfenic acid derivatives, such as captafol, captan, dichlofluanid, folpet, tolylfluanid;
Q) other fungicides, such as acibenzolar-S-methyl, benthiavalicarb, carpropamid, chlorothalonil, cyflufenamid, cymoxanil, dazomet, diclomezin, diclocymet, diclofluanid, diethofencarb, edifenphos, ethaboxam, fenhexamid, fentin-acetate, fenoxanil, ferimzone, fluazinam, fosetyl, fosetyl-aluminum, phosphorous acid, iprovalicarb, hexachlorobenzene, mandipropamid, metrafenone, pencycuron, propamocarb, phthalide, toloclofos-methyl, quintozene, zoxamide,
   oxime ether derivatives of the formula V

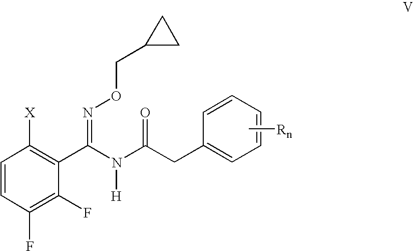

in which
X is C₁-C₄-haloalkoxy,
n is 0, 1, 2 or 3,
R is halogen, C₁-C₄-alkyl, C₁-C₄-haloalkyl, C₁-C₄-alkoxy or haloalkoxy;
phenylamidine derivatives of the formula VI

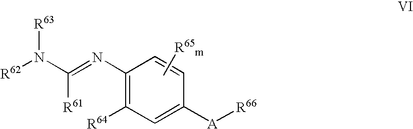

in which the variables are as defined below:
R⁶¹ is hydrogen, C₁-C₈-alkyl, C₂-C₈-alkenyl or C₂-C₈-alkynyl, which are unsubstituted or may be substituted by one to three groups R^{a}:
   R^{a} is halogen, C₁-C₈-alkoxy, C₁-C₈-haloalkoxy, C₁-C₈-alkylthio or phenyl which may be substituted by halogen, C₁-C₈-alkyl, C₁-C₈-haloalkyl, C₁-C₈-alkoxy, C₁-C₈-haloalkoxy or C₁-C₈-alkylthio;
R⁶², R⁶³ can be identical or different and are hydrogen, cyano, C₁-C₈-alkyl, C₂-C₈-alkenyl, C₂-C₈-alkynyl, C₁-C₈-alkoxy, C₁-C₈-alkoxyalkyl, benzyloxy or C₁-C₈-alkylcarbonyl, which are unsubstituted or may be substituted by one to three groups R^{a};
R⁶⁴ is hydrogen, C₁-C₈-alkyl, C₂-C₈-alkenyl or C₂-C₈-alkynyl, which are unsubstituted or may be substituted by one to three groups R^{b}:
   R^{b} is one of the groups mentioned under R^{a}, cyano, C(═O)R^{c}, C(═S)R^{c} or S(O)ₚR^{c},
   R^{c} is C₁-C₈-alkyl, C₁-C₈-haloalkyl, C₁-C₈-alkoxy, C₁-C₈-haloalkoxy, C₁-C₈-alkylthio, amino, C₁-C₈-alkylamino, di(C₁-C₈-alkyl)amino or phenyl which may be substituted by halogen, C₁-C₈-alkyl, C₁-C₈-haloalkyl, C₁-C₈-alkoxy, C₁-C₈-haloalkoxy or C₁-C₈-alkylthio;
m is 0 or 1;
R⁶⁵ is one of the groups mentioned under R⁶⁴;
A is a direct bond, —O—, —S—, NR^{d}, CHR^{e} or —O—CHR^{e}; R^{d}, R^{e} are one of the groups mentioned under R^{a};
R⁶⁶ is phenyl or a five- or six-membered saturated, partially unsaturated or aromatic heterocycle which contains one to four heteroatoms from the group consisting of O, N or S, where the groups R⁶⁶ are unsubstituted or may be substituted by one to three R^{f}:
   R^{f} is one of the groups mentioned under R^{b} or amino, C₁-C₈-alkylamino, di(C₁-C₈-alkyl)amino, C₁-C₈-haloalkyl, C₁-C₈-alkoxyalkyl, C₂-C₈-alkenyloxyalkyl, C₂-C₈-alkynyloxyalkyl, C₁-C₈-alkylcarbonyloxy-C₁-C₈-alkyl, cyanooxy-C₁-C₈-alkyl, C₃-C₆-cycloalkyl or phenoxy, where the cyclic groups may be substituted by halogen, C₁-C₈-alkyl, C₁-C₈-haloalkyl, C₁-C₈-alkoxy, C₁-C₈-haloalkoxy or C₁-C₈-alkylthio;
or
a compound of the formula VII

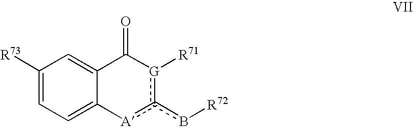

in which the variables are as defined below:
A′ is O or N;
G is C or N;
B is N or a direct bond;
R⁷¹ is C₁-C₄-alkyl;
R⁷² is C₁-C₄-alkoxy; and
R⁷³ is halogen;
R) growth retardants, such as prohexadione and its salts, trinexapac-ethyl, chlormequat, mepiquat-chloride and diflufenzopyr;
   in a synergistically effective amount.

Moreover, the invention relates to novel fungicidal triazolopyrimidines, to a method for controlling harmful fungi using mixtures of a compound I with an active compound of groups A) to R) and to the use of the compounds I with the active compounds of groups A) to R) for preparing such mixtures, and also to compositions comprising such mixtures.

The compounds I referred to above as component 1, their preparation and their action against harmful fungi are proposed in a general manner in the literature (U.S. Pat. No. 5,593,996). Individual compounds of the formula I are known from U.S. Pat. No. 5,593,996; WO 02/02563; WO 02/94020).

Mixtures of 5-chlorotriazolopyrimidines with various active compounds are known in a general manner from EP-A 988 790 and U.S. Pat. No. 6,268,371.

It is an object of the present invention, with a view to reducing the application rates of the known compounds, to provide novel active compounds or mixtures which, at a reduced total amount of active compounds applied, have improved activity against harmful fungi.

Accordingly, the mixtures defined at the outset and novel active compounds have been found. Moreover, it has been found that simultaneous, that is joint or separate, application of a compound I and an active compound II from groups A) to R) or successive application of a compound I and an active compound from groups A) to R) allows better control of harmful fungi than is possible with the individual compounds (synergistic mixtures).

Suitable for use according to the invention are in particular the compounds compiled in the following table, where the groups D and L have the following meanings:

TABLE A

| Com- | | |
|---|---|---|
| pound | | |
| No. | D | L |
| A-1 | —(CH₂)₄— | CH₃ |
| A-2 | —(CH₂)₄— | F |
| A-3 | —(CH₂)₄— | Cl |
| A-4 | —CH(CH₃)—CH₂—CH₂—CH₂— | CH₃ |
| A-5 | —CH(CH₃)—CH₂—CH₂—CH₂— | F |
| A-6 | —CH(CH₃)—CH₂—CH₂—CH₂— | Cl |
| A-7 | —CH₂—CH(CH₃)—CH₂—CH₂— | CH₃ |
| A-8 | —CH₂—CH(CH₃)—CH₂—CH₂— | F |
| A-9 | —CH₂—CH(CH₃)—CH₂—CH₂— | Cl |
| A-10 | —CH(CH₃)—CH(CH₃)—CH₂—CH₂— | CH₃ |
| A-11 | —CH(CH₃)—CH(CH₃)—CH₂—CH₂— | F |
| A-12 | —CH(CH₃)—CH(CH₃)—CH₂—CH₂— | Cl |
| A-13 | —CH(CH₃)—CH₂—CH(CH₃)—CH₂— | CH₃ |
| A-14 | —CH(CH₃)—CH₂—CH(CH₃)—CH₂— | F |
| A-15 | —CH(CH₃)—CH₂—CH(CH₃)—CH₂— | Cl |
| A-16 | —CH₂—CH(CH₃)—CH(CH₃)—CH₂— | CH₃ |
| A-17 | —CH₂—CH(CH₃)—CH(CH₃)—CH₂— | F |
| A-18 | —CH₂—CH(CH₃)—CH(CH₃)—CH₂— | Cl |
| A-19 | —CH(CH₃)—CH₂—CH₂—CH(CH₃)— | CH₃ |
| A-20 | —CH(CH₃)—CH₂—CH₂—CH(CH₃)— | F |
| A-21 | —CH(CH₃)—CH₂—CH₂—CH(CH₃)— | Cl |
| A-22 | —(CH₂)₅— | CH₃ |
| A-23 | —(CH₂)₅— | F |
| A-24 | —(CH₂)₅— | Cl |
| A-25 | —CH(CH₃)—CH₂—CH₂—CH₂—CH₂— | CH₃ |
| A-26 | —CH(CH₃)—CH₂—CH₂—CH₂—CH₂— | F |
| A-27 | —CH(CH₃)—CH₂—CH₂—CH₂—CH₂— | Cl |
| A-28 | —CH₂CH(CH₃)—CH₂—CH₂—CH₂— | CH₃ |
| A-29 | —CH₂CH(CH₃)—CH₂—CH₂—CH₂— | F |
| A-30 | —CH₂CH(CH₃)—CH₂—CH₂—CH₂— | Cl |
| A-31 | —CH₂—CH₂—CH(CH₃)—CH₂—CH₂— | CH₃ |
| A-32 | —CH₂—CH₂—CH(CH₃)—CH₂—CH₂— | F |
| A-33 | —CH₂—CH₂—CH(CH₃)—CH₂—CH₂— | Cl |
| A-34 | —CH(CH₃)—CH(CH₃)—CH₂—CH₂—CH₂— | CH₃ |
| A-35 | —CH(CH₃)—CH(CH₃)—CH₂—CH₂—CH₂— | F |
| A-36 | —CH(CH₃)—CH(CH₃)—CH₂—CH₂—CH₂— | Cl |
| A-37 | —CH(CH₃)—CH₂—CH(CH₃)—CH₂—CH₂— | CH₃ |
| A-38 | —CH(CH₃)—CH₂—CH(CH₃)—CH₂—CH₂— | F |
| A-39 | —CH(CH₃)—CH₂—CH(CH₃)—CH₂—CH₂— | Cl |
| A-40 | —CH(CH₃)—CH₂—CH₂—CH(CH₃)—CH₂— | CH₃ |
| A-41 | —CH(CH₃)—CH₂—CH₂—CH(CH₃)—CH₂— | F |
| A-42 | —CH(CH₃)—CH₂—CH₂—CH(CH₃)—CH₂— | Cl |
| A-43 | —CH(CH₃)—CH₂—CH₂—CH₂—CH(CH₃)— | CH₃ |
| A-44 | —CH(CH₃)—CH₂—CH₂—CH₂—CH(CH₃)— | F |
| A-45 | —CH(CH₃)—CH₂—CH₂—CH₂—CH(CH₃)— | Cl |
| A-46 | —CH₂—CH(CH₃)—CH(CH₃)—CH₂—CH₂— | CH₃ |
| A-47 | —CH₂—CH(CH₃)—CH(CH₃)—CH₂—CH₂— | F |
| A-48 | —CH₂—CH(CH₃)—CH(CH₃)—CH₂—CH₂— | Cl |
| A-49 | —CH(CH₂CH₃)—CH₂—CH₂—CH₂—CH₂— | CH₃ |
| A-50 | —CH(CH₂CH₃)—CH₂—CH₂—CH₂—CH₂— | F |
| A-51 | —CH(CH₂CH₃)—CH₂—CH₂—CH₂—CH₂— | Cl |
| A-52 | —CH—CH(CH₂CH₃)—CH₂—CH₂—CH₂— | CH₃ |
| A-53 | —CH—CH(CH₂CH₃)—CH₂—CH₂—CH₂— | F |
| A-54 | —CH—CH(CH₂CH₃)—CH₂—CH₂—CH₂— | Cl |
| A-55 | —CH₂—CH₂—CH(CH₂CH₃)—CH₂—CH₂— | CH₃ |
| A-56 | —CH₂—CH₂—CH(CH₂CH₃)—CH₂—CH₂— | F |
| A-57 | —CH₂—CH₂—CH(CH₂CH₃)—CH₂—CH₂— | Cl |
| A-58 | —CH₂—CH₂—CH(CH₂CH₂CH₃)—CH₂—CH₂— | CH₃ |
| A-59 | —CH₂—CH₂—CH(CH₂CH₂CH₃)—CH₂—CH₂— | F |
| A-60 | —CH₂—CH₂—CH(CH₂CH₂CH₃)—CH₂—CH₂— | Cl |
| A-61 | —CH₂—CH₂—CH₂—CH(CH[CH₃]₂)—CH₂—CH₂— | CH₃ |
| A-62 | —CH₂—CH₂—CH₂—CH(CH[CH₃]₂)—CH₂—CH₂— | F |
| A-63 | —CH₂—CH₂—CH₂—CH(CH[CH₃]₂)—CH₂—CH₂— | Cl |
| A-64 | —CH₂—CH₂—CH(C[CH₃]₃)—CH₂—CH₂— | CH₃ |
| A-65 | —CH₂—CH₂—CH(C[CH₃]₃)—CH₂—CH₂— | F |
| A-66 | —CH₂—CH₂—CH(C[CH₃]₃)—CH₂—CH₂— | Cl |
| A-67 | —(CH₂)₆— | CH₃ |
| A-68 | —(CH₂)₆— | F |
| A-69 | —(CH₂)₆— | Cl |

Compounds I in which L is methyl and D has one of the meanings below:

—(CH₂)₄—; —CH₂—CH(CH₃)—CH₂—CH₂—; —CH(CH₃)—CH(CH₃)—CH₂—CH₂—; —CH₂—CH(CH₃)—CH(CH₃)—CH₂—; —CH(CH₃)—CH₂—CH(CH₃)—CH₂—; —(CH₂)₅—; —CH(CH₃)—CH₂—CH₂—CH₂—CH₂—; —CH(CH₃)—CH₂—CH₂—CH(CH₃)—; —CH₂—CH₂—CH(CH₃)—CH₂—CH₂—; —CH(CH₃)—CH(CH₃)—CH₂—CH₂—CH₂—; —CH(CH₃)—CH₂—CH(CH₃)—CH₂—CH₂—; —CH(CH₃)—CH₂—CH₂—CH(CH₃)—CH₂—; —CH(CH₃)—CH₂—CH₂—CH₂—CH(CH₃)—; —CH₂—CH(CH₃)—CH(CH₃)—(CH₂)₂—; —CH₂—CH(CH₃)—CH₂—CH(CH₃)—CH₂—; —(CH₂)₂—CH(CH[CH₃]₂)—(CH₂)₂—; —CH(CH₂CH₃)—CH₂—CH₂—CH₂—CH₂—; —CH—CH(CH₂CH₃)—CH₂—CH₂—CH₂—; —CH₂—CH₂—CH(CH₂CH₃)—CH₂—CH₂—; —CH₂—CH₂—CH(CH₂CH₂CH₃)—CH₂—CH₂—; —CH₂—CH₂—CH(C[CH₃]₃)—CH₂—CH₂— and —(CH₂)₆— are novel. They are a preferred subject matter of the invention.

In addition, preference is also given to compounds I in which L is fluorine and D has one of the meanings below:

(CH₂)₄—; —CH₂—CH(CH₃)—CH₂—CH₂—; —CH(CH₃)—CH(CH₃)—CH₂—CH₂—; —CH(CH₃)—CH₂—CH(CH₃)—CH₂—; —CH(CH₃)—CH₂—CH₂—CH(CH₃)—; —CH₂—CH(CH₃)—CH₂—CH₂—CH₂—; —CH₂—CH₂—CH(CH₃)—CH₂—CH₂—; —CH₂—CH(CH₃)—CH(CH₃)—CH₂—; —CH(CH₃)—CH(CH₃)—CH₂—CH₂—CH₂—; —CH(CH₃)—CH₂—CH(CH₃)—CH₂—CH₂—; —CH(CH₃)—CH₂—CH₂—CH(CH₃)—CH₂—; —CH(CH₃)—CH₂—CH₂—CH₂—CH(CH₃)—; —CH₂—CH(CH₃)—CH(CH₃)—(CH₂)₂—; —CH₂—CH(CH₃)—CH₂—CH(CH₃)—CH₂—; —(CH₂)₂—CH(CH[CH₃]₂)—(CH₂)₂—; —CH(CH₂CH₃)—CH₂—CH₂—CH₂—CH₂—; —CH—CH(CH₂CH₃)—CH₂—CH₂—CH₂—; —CH₂—CH₂—CH(CH₂CH₃)—CH₂—CH₂—; —CH₂—CH₂—CH(CH₂CH₂CH₃)—CH₂—CH₂— and —CH₂—CH₂—CH(C[CH₃]₃)—CH₂—CH₂—. These compounds, too, are novel.

In addition, preference is also given to compounds I in which L is chlorine and D has one of the meanings below:

—CH(CH₃)—CH(CH₃)—CH₂—CH₂—; —CH₂—CH(CH₃)—CH₂—CH₂—; —CH(CH₃)—CH₂—CH(CH₃)—CH₂—; —CH(CH₃)—CH₂—CH₂—CH(CH₃)—; —CH₂—CH(CH₃)—CH(CH₃)—CH₂—; —CH(CH₃)—CH(CH₃)—CH₂—CH₂—CH₂—; —CH(CH₃)—CH₂—CH₂—CH(CH₃)—CH₂—; —CH(CH₃)—CH₂—CH₂—CH₂—CH(CH₃)—; —CH₂—CH(CH₃)—CH(CH₃)—(CH₂)₂—; —(CH₂)₂—CH(CH[CH₃]₂)—(CH₂)₂—; —CH(CH₂CH₃)—CH₂—CH₂—CH₂—CH₂—; —CH—CH(CH₂CH₃)—CH₂—CH₂—CH₂—; —CH₂—CH₂—CH(CH₂CH₃)—CH₂—CH₂—; —CH₂—CH₂—CH(CH₂CH₂CH₃)—CH₂—CH₂— and —CH₂—CH₂—CH(C[CH₃]₃)—CH₂—CH₂—. These compounds are novel.

The novel compounds can be obtained by different routes. Advantageously, they are obtained by reacting dichlorotriazolopyrimidines of the formula II′ in which L is methyl, fluorine or chlorine with amines of the formula II′ in which D is as defined for formula I, under conditions known in a general manner from WO 98/46608.

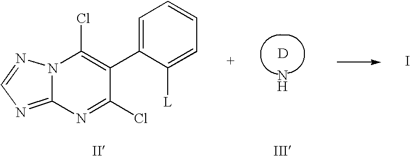

The reaction of II′ with amines-III′ is advantageously carried out at from 0° C. to 70° C., preferably from 10° C. to 35° C., preferably in the presence of an inert solvent, such as an ether, for example dioxane, diethyl ether or, in particular, tetrahydrofuran, a halogenated hydrocarbon, such as dichloromethane or an aromatic hydrocarbon, such as, for example, toluene.

The use of a base, such as a tertiary amine, for example triethylamine or an inorganic base, such as potassium carbonate, is preferred; it is also possible for excess amine of the formula III to serve as base.

Amines of the formula III′ are commercially available.

The active compounds of groups A) to R) mentioned above as component 2, their preparation and their action against harmful fungi are generally known (cf.: http://www.hclrss.demon.co.uk/index.html):
bitertanol, β-([1,1′-biphenyl]-4-yloxy)-α-(1,1-dimethylethyl)-1H-1,2,4-triazol-1-ethanol; bromoconazole, 1-[4-bromo-2-(2,4-dichlorophenyl)tetrahydrofuran-2-ylmethyl]-1H-[1,2,4]triazole (Proc. Br. Crop Prot. Conf.-Pests Dis., 5-6, 439 (1990));
cyproconazole, 2-(4-chlorophenyl)-3-cyclopropyl-1-[1,2,4]triazol-1-ylbutan-2-ol (U.S. Pat. No. 4,664,696);
difenoconazole, 1-{2-[2-chloro-4-(4-chlorophenoxy)phenyl]-4-methyl-[1,3]dioxolan-2-ylmethyl}-1H-[1,2,4]triazole (GB-A 2 098 607);
dinitroconazole, 1-(2,4-dichlorophenyl)-4,4-dimethyl-2-[1,2,4]triazol-1-ylpent-1-en-3-ol (CAS RN [83657-24-3]);
epoxiconazole, (2RS, 3SR)-1-[3-(2-chlorophenyl)-2,3-epoxy-2-(4-fluorophenyl)propyl]-1H-1,2,4-triazole (EP-A 196 038);
fenbuconazole, 3-(4-chlorophenyl)-2-phenyl-2-[1,2,4]triazol-1-ylpropionitrile (EP-A 251 775);
fluquinconazole, 3-(2,4-dichlorophenyl)-6-fluoro-2-[1,2,4]triazol-1-yl-3H-quinazolin-4-one (Proc. Br. Crop Prot. Conf.-Pests Dis., 5-3, 411 (1992));
flusilazole, 1-{[bis-(4-fluorophenyl)methylsilanyl]methyl}-1H-[1,2,4]triazole (Proc. Br. Crop Prot. Conf.-Pests Dis., 1, 413 (1984));
flutriafol, 1-(4-fluorophenyl)-1-(2-fluorophenyl)-2-[1,2,4]triazol-1-ylethanol (CAS RN [76674-21-0]);
hexaconazole, 2-(2,4-dichlorophenyl)-1-[1,2,4]triazol-1-ylhexan-2-ol (CAS RN [79983-714]);
imazalil,
ipconazole, 2-(4-chlorobenzyl)-5-isopropyl-1-[1,2,4]triazol-1-ylmethylcyclopentanol (EP-A 267 778);
metconazole, 5-(4-chlorobenzyl)-2,2-dimethyl-1-[1,2,4]triazol-1-ylmethylcyclopentanol (GB 857 383);
myclobutanil, 2-(4-chlorophenyl)-2-[1,2,4]triazol-1-ylmethylpentanenitrile (CAS RN [88671-89-0]);
penconazole, 1-[2-(2,4-dichlorophenyl)pentyl]-1H-[1,2,4]triazole (Pesticide Manual, 12th Ed. (2000), page 712);
propiconazole, 1-[2-(2,4-dichlorophenyl)-4-propyl-[1,3]dioxolan-2-ylmethyl]-1H-[1,2,4]triazole (GB 15 22 657);
prochloraz, N-propyl-N-[2-(2,4,6-trichlorophenoxy)ethyl]imidazole-1-carboxamide (U.S. Pat. No. 3,991,071);
prothioconazole, 2-[2-(1-chlorocyclopropyl)-3-(2-chlorophenyl)-2-hydroxypropyl]-2,4-dihydro-[1,2,4]triazole-3-thione (WO 96/16048);
simeconazole, 1-(4-fluorophenyl)-2-[1,2,4]triazol-1-yl-1-trimethylsilanylethanol (The BCPC Conference Pests and Diseases 2000, p. 557-562);
tebuconazole, 1-(4-chlorophenyl)-4,4-dimethyl-3-[1,2,4]triazol-1-ylmethylpentan-3-ol (EP-A 40 345);
tetraconazole, 1-[2-(2,4-dichlorophenyl)-3-(1,1,2,2-tetrafluoroethoxy)propyl]-1H-[1,2,4]triazole (Proc. Br. Crop Prot. Conf.-Pests Dis., 1, 49 (1988));
triadimefon, 1-(4-chlorophenoxy)-3,3-dimethyl-1-(1H-1,2,4-triazol-1-yl)-2-butanone;
triadimenol, β-(4-chlorophenoxy)-α-(1,1-dimethylethyl)-1H-1,2,4-triazol-1-ethanol;
triflumizole, (4-chloro-2-trifluoromethylphenyl)(2-propoxy-1-[1,2,4]triazol-1-ylethyli-dene)amine (JP-A 79/119 462);
triticonazole, 5-(4-chlorobenzylidene)-2,2-dimethyl-1-[1,2,4]triazol-1-ylmethylcyclo-pentanol (EP-A 378 953);
azoxystrobin, methyl 2-{2-[6-(2-cyano-1-vinylpenta-1,3-dienyloxy)pyrimidin-4-yloxy]phenyl}-methoxyacrylate (EP-A 382 375)
dimoxystrobin, (E)-2-(methoxyimino)-N-methyl-2-[α-(2,5-xylyloxy)-o-tolyl]acetamide (EP-A 477 631);
fluoxastrobin, (E)-{2-[6-(2-chlorophenoxy)-5-fluoropyrimidin-4-yloxy]phenyl}(5,6-dihydro-1,4,2-dioxazin-3-yl)methanone O-methyloxime (WO 97/27189);
kresoxim-methyl, methyl (E)-methoxyimino[α-(otolyloxy)-otolyl]acetate (EP-A 253 213);
metominostrobin, (E)-2-(methoxyimino)-N-methyl-2-(2-phenoxyphenyl)acetamide (EP-A 398 692);
orysastrobin, (2E)-2-(methoxyimino)-2-{2-[(3E,5E,6E)-5-(methoxyimino)-4,6-dimethyl-2,8-dioxa-3,7-diazanona-3,6-dien-1-yl]phenyl}-N-methylacetamide (WO 97/15552);
picoxystrobin, methyl 3-methoxy-2-[2-(6-trifluoromethylpyridin-2-yloxymethyl)phenyl]-acrylate (EP-A 278 595);
pyraclostrobin, methyl N-{2-[1-(4-chlorophenyl)-1H-pyrazol-3-yloxymethyl]phenyl}(N-methoxy)carbamate (WO-A 96/01256);
trifloxystrobin, methyl (E)-methoxyimino-{(E)-α-[1-(α,α,α-trifluoro-m-tolyl)ethylidene-aminooxy]-otolyl}acetate (EP-A 460 575);
methyl 2-(ortho-(2,5-dimethylphenyloxymethylene)phenyl)-3-methoxyacrylate (EP-A 226 917);
benalaxyl,
metalaxyl, methyl N-(methoxyacetyl)-N-(2,6-xylyl)-DL-alaninate (GB 15 00 581);
mefenoxam, methyl N-(methoxyacetyl)-N-(2,6-xylyl)-D-alaninate (WO 96/01559);
ofurace, (RS)-α-(2-chloro-N-2,6-xylylacetamido)-γ-butyrolactone;
oxadixyl; N-(2,6-dimethylphenyl)-2-methoxy-N-(2-oxo-3-oxazolidinyl)acetamide;
aldimorph, 4-alkyl-2,5(or 2,6)-dimethylmorpholine, comprising 65-75% 2,6-dimethyl-morpholine and 25-35% 2,5-dimethylmorpholine, more than 85% being 4-dodecyl-2,5(or 2,6)-dimethylmorpholine and where “alkyl” may also be octyl, decyl, tetradecyl or hexadecyl and where the cis/trans ratio is 1:1;
dodine, (2,4-dichlorophenoxy)acetic acid (U.S. Pat. No. 2,867,562);
dodemorph, 4-cyclododecyl-2,6-dimethylmorpholine (DE 11 98 125);
fenpropimorph, 4-[3-(4-tert-butylphenyl)-2-methylpropyl]-2,6-dimethylmorpholine (DE 26 56 747);
fenpropidin, 1-[3-(4-tert-butylphenyl)-2-methylpropyl]piperidine (DE-A 27 52 096);
guazatine, mixture comprising iminoctadine, bis(8-guanidino-octyl)amine (GB 1114155);
spiroxamine, (8-tert-butyl-1,4-dioxaspiro[4,5]dec-2-yl)diethylamine (EP-A 281 842); tridemorph, mixture of N-alkylmorpholine derivatives which comprises 2,6-dimethyl-4-tridecylmorpholine as main component (DE-B 11 64 152);
pyrimethanil, 4,6-dimethylpyrimidin-2-ylphenylamine (DD-A 151 404);
mepanipyrim, (4-methyl-6-prop-1-ynylpyrimidin-2-yl)phenylamine (EP-A 224 339);
cyprodinil, (4-cyclopropyl-6-methylpyrimidin-2-yl)phenylamine (EP-A 310 550);
iprodione, N-isopropyl-3-(3,5-dichlorophenyl)-2,4-dioxoimidazolidine-1-carboxamide (GB 13 12 536);
myclozolin, (RS)-3-(3,5-dichlorophenyl)-5-methoxymethyl-5-methyl-1,3-oxazolidine-2,4-dione;
procymidone, N-(3,5-dichlorophenyl)-1,2-dimethylcyclopropane-1,2-dicarboximide (U.S. Pat. No. 3,903,090);
vinclozolin, 3-(3,5-dichlorophenyl)-5-methyl-5-vinyloxazolidine-2,4-dione (DE-A 22 07 576);
dimethomorph, 3-(4-chlorophenyl)-3-(3,4-dimethoxyphenyl)-1-morpholin-4-ylpropenone (EP-A 120 321);
flumetover
flumorph, 3-(4-fluorophenyl)-3-(3,4-dimethoxyphenyl)-1-morpholin-4-ylpropenone (EP-A 860 438);
cycloheximide, 4-{(2R)-2-[(1S,3S,5S)-3,5-dimethyl-2-oxocyclohexyl]-2-hydroxyethyl}pi-peridine-2,6-dione;
griseofulvin, 7-chloro-2′,4,6-trimethoxy-6′-methylspiro[benzofuran-2(3H),1′-cyclohex-2′-ene]-3,4′-dione,
kasugamycin, 1 L-1,3,4/2,5,6-1-deoxy-2,3,4,5,6-pentahydroxycyclohexyl-2-amino-2,3,4,6-tetradeoxy-4-(α-iminoglycino)-α-D-arabinohexopyranoside;
natamycin, (8E,14E,16E,18E,20E)-(1R,3S,5R,7R,12R,22R,24S,25R,26S)-22-(3-amino-3,6-dideoxy-β-D-mannopyranosyloxy)-1,3,26-trihydroxy-12-methyl-10-oxo-6,11,28-trioxatricyclo[22.3.1.0⁵⁷]octacosa-8,14,16,18,20-pentaene-25-carboxylic acid;
polyoxin, 5-(2-amino-5-O-carbamoyl-2-deoxy-L-xylonamido)-1-(5-carboxy-1,2,3,4-te-trahydro-2,4-dioxopyrimidin-1-yl)-1,5-dideoxy-β-D-allofuranuronic acid and its salts;
streptomycin, O-2-deoxy-2-methylamino-α-L-glucopyranosyl-(1→2)—O-5-deoxy-3-C-formyl-α-L-lyxofuranosyl-(1→4)—N¹N³-diamidino-D-streptamine;
ferbam, iron(3+) dimethyldithiocarbamate (U.S. Pat. No. 1,972,961);
nabam, disodium ethylenebis(dithiocarbamate) (U.S. Pat. No. 2,317,765);
maneb, manganese ethylenebis(dithiocarbamate) (U.S. Pat. No. 2,504,404);
mancozeb, manganese ethylenebis(dithiocarbamate) zinc complex (U.S. Pat. No. 3,379,610);
metam, methyldithiocarbamic acid (U.S. Pat. No. 2,791,605)
metiram, zinc ammoniate ethylenebis(dithiocarbamate) (U.S. Pat. No. 3,248,400);
propineb, zinc propylenebis(dithiocarbamate) polymer (BE 611 960);
polycarbamate,
thiram, bis(dimethylthiocarbamoyl) disulfide (DE 642 532);
ziram, dimethyldithiocarbamate;
zineb, zinc ethylenebis(dithiocarbamate) (U.S. Pat. No. 2,457,674);
anilazine, 4,6-dichloro-N-(2-chlorophenyl)-1,3,5-triazine-2-amine (U.S. Pat. No. 2,720,480);
benomyl, N-butyl-2-acetylaminobenzoimidazole-1-carboxamide (U.S. Pat. No. 3,631,176);
boscalid, 2-chloro-N-(4′-chlorobiphenyl-2-yl)nicotinamide (EP-A 545 099);
carbendazim, methyl (1H-benzimidazol-2-yl)carbamate (U.S. Pat. No. 3,657,443);
carboxin, N-phenyl-2-methyl-5,6-dihydro-[1,4]oxathiine-3-carboxamide (U.S. Pat. No. 3,454,391);
oxycarboxin, 5,6-dihydro-2-methyl-1,4-oxathiine-3-carboxanilide 4,4-dioxide (U.S. Pat. No. 3,454,391);
cyazofamid, 4-chloro-2-cyano-N,N-dimethyl-5-p-tolylimidazole-1-sulfonamide (EP-A 298 196)
dazomet, 3,5-dimethyl-1,3,5-thiadiazinane-2-thione;
dithianon, 5,10-dioxo-5,10-dihydronaphtho[2,3-b][1,4]dithiine-2,3-dicarbonitrile (GB 857 383);
famoxadone, (RS)-3-anilino-5-methyl-5-(4-phenoxyphenyl)-1,3-oxazolidine-2,4-dione;
fenamidone, (S)-1-anilino-4-methyl-2-methylthio-4-phenylimidazolin-5-one;
fenarimol, (4-chlorophenyl)(2-chlorophenyl)pyrimidin-5-ylmethanol (GB 12 18 623);
fuberidazole, 2-(2-furanyl)-1H-benzimidazole (DE 12 09 799);
flutolanil, N-(3-isopropoxyphenyl)-2-trifluoromethylbenzamide (JP 1104514);
furametpyr, (RS)-5-chloro-N-(1,3-dihydro-1,1,3-trimethylisobenzofuran-4-yl)-1,3-dime-thylpyrazole-4-carboxamide;
isoprothiolane, indol-3-ylacetic acid;
mepronil, 3′-isopropoxy-otoluanilide (U.S. Pat. No. 3,937,840);
nuarimol, α-(2-chlorophenyl)-α-(4-fluorophenyl)-5-pyrimidinemethanol (GB 12 18 623);
picobenzamid, 2,6-dichloro-N-(3-chloro-5-trifluoromethylpyridin-2-ylmethyl)benzamide (WO 99/42447);
probenazole, 3-allyloxy-1,2-benzothiazole 1,1-dioxide;
proquinazid, 6-iodo-2-propoxy-3-propylquinazolin-4(3H)-one (WO 97/48684);
pyrifenox, 1-(2,4-dichlorophenyl)-2-(3-pyridinyl)ethanone O-methyloxime (EP-A 49 854);
pyroquilon, 1,2,5,6-tetrahydropyrrolo[3,2,1-ij]quinolin-4-one (GB 13 94 373);
quinoxyfen, 5,7-dichloro-4-(4-fluorophenoxy)quinoline (U.S. Pat. No. 5,240,940);
silthiofam, N-allyl-4,5-dimethyl-2-(trimethylsilyl)thiophene-3-carboxamide;
thiabendazole, 2-(thiazol-4-yl)benzimidazole (U.S. Pat. No. 3,017,415);
thifluzamide, N-[2,6-d]bromo-4-(trifluoromethoxy)phenyl]-2-methyl-4-(trifluoromethyl)-5-thiazolecarboxamide;
thiophanate-methyl, dimethyl 1,2-phenylenebis(iminocarbonothioyl)bis(carbamate) (DE-A 19 30 540);
tiadinil, 3′-chloro-4,4′-dimethyl-1,2,3-thiadiazole-5-carboxanilide;
tricyclazole, 5-methyl-1,2,4-triazolo[3,4-b]benzothiazole (GB 14 19 121);
triforine, N,N′-{piperazine-1,4-diylbis[(trichloromethyl)methylene]}diformamide (DE 19 01 421);
3-[5-(4-chlorophenyl)-2,3-dimethylisoxazolidin-3-yl]pyridine (EP-A 1035 122);
methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate (EP-A 1201 648);
sulfur;

Bordeaux mixture, the mixture of calcium hydroxide and copper(II) sulfate;

copper acetate; copper oxychloride; basic copper sulfate;
binapacryl, (RS)-2-sec-butyl-4,6-dinitrophenyl 3-methylcrotonate;
dinocap, the mixture of 2,6-dinitro-4-octylphenyl crotonate and 2,4-dinitro-6-octylphenyl crotonate, where “octyl” is a mixture of 1-methylheptyl, 1-ethylhexyl and 1-propylpentyl (U.S. Pat. No. 2,526,660);
dinobuton, (RS)-2-sec-butyl-4,6-dinitrophenyl isopropyl carbonate;
nitrophthal-isopropyl; diisopropyl 5-nitroisophthalate;
fenpiclonil, 4-(2,3-dichlorophenyl)-1H-pyrrole-3-carbonitrile (Proc. 1988 Br. Crop Prot. Conf.- Pests Dis., Vol. 1, p. 65);
fludioxonil, 4-(2,2-difluorobenzo[1,3]dioxol-4-yl)-1H-pyrrole-3-carbonitrile (The Pesticide Manual, pubi. The British Crop Protection Council, 10th Ed. (1995), p. 482);
captafol, N-(1,1,2,2-tetrachloroethylthio)cyclohex-4-ene-1,2-dicarboximide;
captan, 2-trichloromethylsulfanyl-3a,4,7,7a-tetrahydroisoindole-1,3-dione (U.S. Pat. No. 2,553,770);
dichlofluanid, N-dichlorofluoromethylthio-N′,N′-dimethyl-N-phenylsulfamide (DE 11 93 498);
folpet, 2-trichloromethylsulfanylisoindole-1,3-dione (U.S. Pat. No. 2,553,770);
tolylfluanid, N-dichlorofluoromethylthio-N′,N′-dimethyl-N-p-tolylsulfamide (DE 11 93 498);
acibenzolar-S-methyl, methyl benzo[1,2,3]thiadiazole-7-carbothioate;
benthiavalicarb, isopropyl {(S)-1-[(1R)-1-(6-fluorobenzothiazol-2-yl)ethylcarbamoyl]-2-methylpropyl}carbamate (JP-A 09/323,984);
carpropamid, 2,2-dichloro-N-[1-(4-chlorophenyl)ethyl]-1-ethyl-3-methylcyclopropane-carboxamide;
chlorothalonil, 2,4,5,6-tetrachloroisophthalonitrile (U.S. Pat. No. 3,290,353);
cyflufenamid, (Z)-N-[α-(cyclopropylmethoxyimino)-2,3-difluoro-6-(trifluoromethyl)benzyl]-2-phenylacetamide (WO 96/19442);
cymoxanil, 1-(2-cyano-2-methoxyiminoacetyl)-3-ethylurea (U.S. Pat. No. 3,957,847);
diclomezin, 6-(3,5-dichlorophenyl-p-tolyl)pyridazin-3(2H)-one;
diclocymet, 2-cyano-N-[(1R)-1-(2,4-dichlorophenyl)ethyl]-3,3-dimethylbutanamide;
diclofluanid, N-dichlorofluoromethylthio-N′,N′-dimethyl-N-phenylsulfonamide (DE-B 11 93 498);
diethofencarb, isopropyl 3,4-diethoxycarbanilate;
edifenphos, O-ethyl S,S-diphenyl phosphorodithioate;
ethaboxam, N-(cyano-2-thienylmethyl)-4-ethyl-2-(ethylamino)-5-thiazolecarboxamide;
fenhexamid, N-(2,3-dichloro-4-hydroxyphenyl)-1-methylcyclohexanecarboxamide (Proc. Br. Crop Prot. Conf.-Pests Dis., 1998, Vol. 2, p. 327);
fentin-acetate, triphenyltin acetate;
fenoxanil, N-(1-cyano-1,2-dimethylpropyl)-2-(2,4-dichlorophenoxy)propanamide;
ferimzone, (Z)-2′-methylacetophenone 4,6-dimethylpyrimidin-2-ylhydrazone;
fluazinam, 3-chloro-N-[3-chloro-2,6-dinitro-4-(trifluoromethyl)phenyl]-5-(trifluoromethyl)-2-pyridineamine (The Pesticide Manual, Publ. The British Crop Protection Council, 10th Ed. (1995), p. 474);
phosphorous acid, fosetyl, fosetyl-aluminum, (aluminum) ethyl phosphonate (FR 22 54 276);
iprovalicarb, isopropyl[(1S)-2-methyl-1-(1-p-tolylethylcarbamoyl)propyl]carbamate (EP-A 472 996);
hexachlorobenzene;
mandipropamid, 4-chloro-N-[2-[3-methoxy-4-(2-propynyloxy)phenyl]ethyl]-α-(2-propynyloxy)benzylacetamide (WO 03/042166);
metrafenone, 3′-bromo-2,3,4,6′-tetramethoxy-2′,6-dimethylbenzophenone (U.S. Pat. No. 5,945,567);
pencycuron, 1-(4-chlorobenzyl)-1-cyclopentyl-3-phenylurea (DE 27 32 257);
propamocarb, propyl 3-(dimethylamino)propylcarbamate (DE 16 43 040);
phthalide,
tolciofos-methyl, O-2,6-dichloro-p-tolyl O,O-dimethyl phosphorothioate (GB 14 67 561);
quintozene, pentachloronitrobenzene (DE 682 048);
zoxamide, (RS)-3,5-dichloro-N-(3-chloro-1-ethyl-1-methyl-2-oxopropyl)-p-toluamide;
prohexadione and its salts (EP-A 123 001),
trinexapac-ethyl, ethyl 4-cyclopropyl(hydroxy)methylene-3,5-dioxocyclohexane-carboxylate (EP-A 126 713);
chlormequat, 2-chloroethyltrimethylammonium salt (U.S. Pat. No. 3,395,009);
mepiquat-chloride, 1,1-dimethylpiperidinium chloride (DE 22 07 575);
diflufenzopyr, 2-{1-[4-(3,5-difluorophenyl)semicarbazono]ethyl}nicotinic acid;
benzoimidazole derivatives of the formula IIA (cf. EP-A 10 17 671);
sulfamoyl compounds of the formula III (cf. EP-A 10 31 571; JP-A 2001-192 381);
thiophene derivatives of the formula IV (cf. JP 10130268);
oxime ether derivatives of the formula V (cf. WO 99/14188);
phenylamidine derivatives of the formula VI (cf. WO 00/46184);
compounds of the formula VII (cf. WO 97/48684; WO 02/094797; WO 03/14103).

The mixtures of the compounds I and the active compounds from groups A) to R) or the simultaneous, that is joint or separate, use of the compounds I and the active compounds from groups A) to R) are distinguished by being highly active against a wide range of phytopathogenic fungi, in particular from the classes of the Ascomycetes, Deuteromycetes, Oomycetes and Basidiomycetes. Some of them act systemically and can be used in crop protection as foliar fungicides, as fungicides for seed dressing and as soil-acting fungicides.

They are particularly important for controlling a multitude of fungi on various cultivated plants, such as bananas, cotton, vegetable species (for example cucumbers, beans and cucurbits), barley, grass, oats, coffee, potatoes, corn, fruit species, rice, rye, soya, tomatoes, grapevines, wheat, ornamental plants, sugar cane and on a large number of seeds.

They are particularly suitable for the control of the following phytopathogenic fungi: Blumeria graminis (powdery mildew) on cereals, Erysiphe cichoracearum and Sphaerotheca fuliginea on cucurbits, Podosphaera leucotricha on apples, Uncinula necator on grapevines, Puccinia species on cereals, Rhizoctonia species on cotton, rice and lawns, Ustilago species on cereals and sugar cane, Venturia inaequalis on apples, Bipolaris and Drechslera species on cereals, rice and lawns, Septoria species on wheat, Botrytis cinerea on strawberries, vegetables, ornamental plants and grapevines, Mycosphaerella species on bananas, peanuts and cereals, Pseudocercosporella herpotrichoides on wheat and barley, Pyricularia oryzae on rice, Phakopsora species on soybean, Phytophthora infestans on potatoes and tomatoes, Pseudoperonospora species on cucurbits and hops, Plasmopara viticola on grapevines, Alternaria species on fruit and vegetables and also Fusarium and Verticillium species.

They can also be used in the protection of materials (e.g. the protection of wood), for example against Paecilomyces variotii.

The compound I and the active compounds from groups A) to R) can be applied simultaneously, that is jointly or separately, or in succession, the sequence, in the case of separate application, generally not having any effect on the result of the control measures.

In the definitions of the symbols given in the formulae above, collective terms were used which are generally representative of the following substituents:

halogen: fluorine, chlorine, bromine and iodine;
alkyl: saturated straight-chain or branched hydrocarbon radicals having 1 to 4, 6, 8 or 10 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-me-thylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dime-thylbutyl, 3,3-dimethylbutyl, 1-ethylbutyl, 2-ethylbutyl, 1,1,2-trimethylpropyl, 1,2,2-tri-methylpropyl, 1-ethyl-1-methylpropyl and 1-ethyl-2-methylpropyl;
haloalkyl: straight-chain or branched alkyl groups having 1 to 2, 4 or 6 carbon atoms (as mentioned above), where some or all of the hydrogen atoms in these groups may be replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
alkenyl: unsaturated straight-chain or branched hydrocarbon radicals having 2 to 4, 6, 8 or 10 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-3-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-bu-tenyl, 2,2-dimethyl-3-butenyl, 2,3-dimethyl-1-butenyl, 2,3-dimethyl-2-butenyl, 2,3-dime-thyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-pro-penyl and 1-ethyl-2-methyl-2-propenyl;
haloalkenyl: unsaturated, straight-chain or branched hydrocarbon radicals having 2 to 10 carbon atoms and one or two double bonds in any position (as mentioned above), where some or all of the hydrogen atoms in these groups may be replaced by halogen atoms as mentioned above, in particular by fluorine, chlorine and bromine;
alkynyl: straight-chain or branched hydrocarbon groups having 2 to 4, 6, 8 or 10 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
cycloalkyl: mono- or bicyclic, saturated hydrocarbon groups having 3 to 6 or 8 carbon ring members, for example C₃-C₈-cycloalkyl, such as cyclopropyl, cyclobutyl, cyclopentyl, cyclohexyl, cycloheptyl and cyclooctyl;
five- to ten-membered saturated, partially unsaturated or aromatic heterocycle which contains one to four heteroatoms from the group consisting of O, N and S:
5- or 6-membered heterocyclyl which contains one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetra-hydrothienyl, 2-pyrrolidinyl, 3-pyrrolidinyl, 3-isoxazolidinyl, 4-isoxazolidinyl, 5-isoxazoli-dinyl, 3-isothiazolidinyl, 4-isothiazolidinyl, 5-isothiazolidinyl, 3-pyrazolidinyl, 4-pyrazoli-dinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahy-dropyridazinyl, 4-hexahydropyridazinyl, 2-hexahydropyrimidinyl, 4-hexahydropyrimi-dinyl, 5-hexahydropyrimidinyl and 2-piperazinyl;
5-membered heteroaryl which contains one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;
6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl;
   alkylene: divalent unbranched chains of 3 to 5 CH₂ groups, for example CH₂, CH₂CH₂, CH₂CH₂CH₂, CH₂CH₂CH₂CH₂ and CH₂CH₂CH₂CH₂CH₂;
   oxyalkylene: divalent unbranched chains of 2 to 4 CH₂ groups, where one valency is attached to the skeleton via an oxygen atom, for example OCH₂CH₂, OCH₂CH₂CH₂ and OCH₂CH₂CH₂CH₂;
   oxyalkyleneoxy: divalent unbranched chains of 1 to 3 CH₂ groups, where both valencies are attached to the skeleton via an oxygen atom, for example OCH₂O, OCH₂CH₂O and OCH₂CH₂CH₂O.

Formula IIA represents compounds in which Y is bromine (II-1) or chlorine (II-2).

Formula III represents in particular compounds in which the index n is 0, 1 or 2, preferably 0 or 1.

If n is 1, the group R³³ is preferably located in the 5- or 6-position. These compounds are particularly suitable for use in the mixtures according to the invention.

In addition, preference is also given to compounds III in which R³¹ is hydrogen, methyl, ethyl, n- or isopropyl, fluorine, chlorine, bromine, iodine, methylthio, ethylthio, trifluoromethyl, pentafluoroethyl, cyano, phenyl or formyl.

Preference is likewise given to compounds III in which R³² is hydrogen, fluorine, chlorine, bromine, iodine, methyl, trifluoromethyl, methoxycarbonyl, ethoxycarbonyl, isopropoxycarbonyl or n-butoxycarbonyl.

Preference is furthermore given to compounds III in which R³³ is fluorine, chlorine, bromine, iodine, cyano, nitro, methyl, ethyl, methoxy, trifluoromethyl, in particular fluorine, chlorine, bromine, cyano, methyl, trifluoromethyl or methoxy.

In particular with a view to their use in the mixtures according to the invention, preference is given to the compounds III compiled in the tables below.

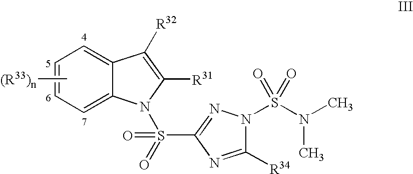

### Table 1

Compounds of the formula III in which n is zero and R³⁴ is hydrogen and the combination of R³¹ and R³² for one compound corresponds in each case to one row of Table III

### Table 2

Compounds of the formula III in which n is zero and R³⁴ is fluorine and the combination of R³¹ and R³² for one compound corresponds in each case to one row of Table III

### Table 3

Compounds of the formula III in which n is zero and R³⁴ is chlorine and the combination of R³¹ and R³² for one compound corresponds in each case to one row of Table III

### Table 4

Compounds of the formula III in which n is zero and R³⁴ is methyl and the combination of R³¹ and R³² for one compound corresponds in each case to one row of Table III

### Table 5

Compounds of the formula III in which n is 1, R³³ is 4-fluoro and R³⁴ is hydrogen and the combination of R³¹ and R³² for one compound corresponds in each case to one row of Table III

### Table 6

Compounds of the formula III in which n is 1, R³³ is 5-fluoro and R³⁴ is hydrogen and the combination of R³¹ and R³² for one compound corresponds in each case to one row of Table III

### Table 7

Compounds of the formula III in which n is 1, R³³ is 6-fluoro and R³⁴ is hydrogen and the combination of R³¹ and R³² for one compound corresponds in each case to one row of Table III

### Table 8

Compounds of the formula III in which n is 1, R³³ is 7-fluoro and R³⁴ is hydrogen, and the combination of R³¹ and R³² for one compound corresponds in each case to one row of Table III

### Table 9

Compounds of the formula III in which n is 1, R³³ is 4-chloro and R³⁴ is hydrogen, and the combination of R³¹ and R³² for one compound corresponds in each case to one row of Table III

### Table 10

Compounds of the formula III in which-n is 1, R³³ is 5-chloro and R³⁴ is hydrogen, and the combination of R³¹ and R³² for one compound corresponds in each case to one row of Table III

### Table 11

Compounds of the formula III in which n is 1, R³³ is 6-chloro and R³⁴ is hydrogen, and the combination of R³¹ and R³² for one compound corresponds in each case to one row of Table III

### Table 12

Compounds of the formula II in which n is 1, R³³ is 7-chloro and R³⁴ is hydrogen, and the combination of R³¹ and R³² for one compound corresponds in each case to one row of Table III

### Table 13

Compounds of the formula III in which n is 1, R³³ is 4-bromo and R³⁴ is hydrogen, and the combination of R³¹ and R³² for one compound corresponds in each case to one row of Table III

### Table 14

Compounds of the formula III in which n is 1, R³³ is 5-bromo and R³⁴ is hydrogen, and the combination of R³¹ and R³² for one compound corresponds in each case to one row of Table III

### Table 15

Compounds of the formula III in which n is 1, R³³ is 6-bromo and R³⁴ is hydrogen, and the combination of R³¹ and R³² for one compound corresponds in each case to one row of Table III

### Table 16

Compounds of the formula III in which n is 1, R³³ is 7-bromo and R³⁴ is hydrogen, and the combination of R³¹ and R³² for one compound corresponds in each case to one row of Table III

### Table 17

Compounds of the formula III in which n is 1, R³³ is 4-iodo and R³⁴ is hydrogen, and the combination of R³¹ and R³² for one compound corresponds in each case to one row of Table III

### Table 18

Compounds of the formula III in which n is 1, R³³ is 5-iodo and R³⁴ is hydrogen, and the combination of R³¹ and R³² for one compound corresponds in each case to one row of Table III

### Table 19

Compounds of the formula III in which n is 1, R³³ is 6-iodo and R³⁴ is hydrogen, and the combination of R³¹ and R³² for one compound corresponds in each case to one row of Table III

### Table 20

Compounds of the formula III in which n is 1, R³³ is 7-iodo and R³⁴ is hydrogen, and the combination of R³¹ and R³² for one compound corresponds in each case to one row of Table III

### Table 21

Compounds of the formula III in which n is 1, R³³ is 4-methyl and R³⁴ is hydrogen, and the combination of R³¹ and R³² for one compound corresponds in each case to one row of Table III

### Table 22

Compounds of the formula III in which n is 1, R³³ is 5-methyl and R³⁴ is hydrogen, and the combination of R³¹ and R³² for one compound corresponds in each case to one row of Table III

### Table 23

Compounds of the formula III in which n is 1, R³³ is 6-methyl and R³⁴ is hydrogen, and the combination of R³¹ and R³² for one compound corresponds in each case to one row of Table III

### Table 24

Compounds of the formula III in which n is 1, R³³ is 7-methyl and R³⁴ is hydrogen, and the combination of R³¹ and R³² for one compound corresponds in each case to one row of Table III

### Table 25

Compounds of the formula III in which n is 1, R³³ is 4-ethyl and R³⁴ is hydrogen, and the combination of R³¹ and R³² for one compound corresponds in each case to one row of Table III

### Table 26

Compounds of the formula III in which n is 1, R³³ is 5-ethyl and R³⁴ is hydrogen, and the combination of R³¹ and R³² for one compound corresponds in each case to one row of Table III

### Table 27

Compounds of the formula III in which n is 1, R³³ is 6-ethyl and R³⁴ is hydrogen, and the combination of R³¹ and R³² for one compound corresponds in each case to one row of Table III

### Table 28

Compounds of the formula III in which n is 1, R³³ is 7-ethyl and R³⁴ is hydrogen, and the combination of R³¹ and R³² for one compound corresponds in each case to one row of Table III

### Table 29

Compounds of the formula III in which n is 1, R³³ is 4-methoxy and R³⁴ is hydrogen, and the combination of R³¹ and R³² for one compound corresponds in each case to one row of Table III

### Table 30

Compounds of the formula III in which n is 1, R³³ is 5-methoxy and R³⁴ is hydrogen, and the combination of R³¹ and R³² for one compound corresponds in each case to one row of Table III

### Table 31

Compounds of the formula III in which n is 1, R³³ is 6-methoxy and R³⁴ is hydrogen, and the combination of R³¹ and R³² for one compound corresponds in each case to one row of Table III

### Table 32

Compounds of the formula III in which n is 1, R³³ is 7-methoxy and R³⁴ is hydrogen, and the combination of R³¹ and R³² for one compound corresponds in each case to one row of Table III

### Table 33

Compounds of the formula III in which n is 1, R³³ is 4-nitro and R³⁴ is hydrogen, and the combination of R³¹ and R³² for one compound corresponds in each case to one row of Table III

### Table 34

Compounds of the formula III in which n is 1, R³³ is 5-nitro and R³⁴ is hydrogen, and the combination of R³¹ and R³² for one compound corresponds in each case to one row of Table III

### Table 35

Compounds of the formula III in which n is 1, R³³ is 6-nitro and R³⁴ is hydrogen, and the combination of R³¹ and R³² for one compound corresponds in each case to one row of Table III

### Table 36

Compounds of the formula III in which n is 1, R³³ is 7-nitro and R³⁴ is hydrogen, and the combination of R³¹ and R³² for one compound corresponds in each case to one row of Table III

### Table 37

Compounds of the formula III in which n is 1, R³³ is 4-cyano and R³⁴ is hydrogen, and the combination of R³¹ and R³² for one compound corresponds in each case to one row of Table III

### Table 38

Compounds of the formula III in which n is 1, R³³ is 5-cyano and R³⁴ is hydrogen, and the combination of R³¹ and R³² for one compound corresponds in each case to one row of Table III

### Table 39

Compounds of the formula III in which n is 1, R³³ is 6-cyano and R³⁴ is hydrogen, and the combination of R³¹ and R³² for one compound corresponds in each case to one row of Table III

### Table 40

Compounds of the formula III in which n is 1, R³³ is 7-cyano and R³⁴ is hydrogen, and the combination of R³¹ and R³² for one compound corresponds in each case to one row of Table III

### Table 41

Compounds of the formula III in which n is 1, R³³ is 4-trifluoromethyl and R³⁴ is hydrogen, and the combination of R³¹ and R³² for one compound corresponds in each case to one row of Table III

### Table 42

Compounds of the formula III in which n is 1, R³³ is 5-trifluoromethyl and R³⁴ is hydrogen, and the combination of R³¹ and R³² for one compound corresponds in each case to one row of Table III

### Table 43

Compounds of the formula III in which n is 1, R³³ is 6-trifluoromethyl and R³⁴ is hydrogen, and the combination of R³¹ and R³² for one compound corresponds in each case to one row of Table III

### Table 44

Compounds of the formula III in which n is 1, R³³ is 7-trifluoromethyl and R³⁴ is hydrogen, and the combination of R³¹ and R³² for one compound corresponds in each case to one row of Table III

### Table 45

Compounds of the formula III in which n is 1, R³³ is 4-methoxycarbonyl and R³⁴ is hydrogen, and the combination of R³¹ and R³² for one compound corresponds in each case to one row of Table III

### Table 46

Compounds of the formula III in which n is 1, R³³ is 5-methoxycarbonyl and R³⁴ is hydrogen, and the combination of R³¹ and R³² for one compound corresponds in each case to one row of Table I

### Table 47

Compounds of the formula III in which n is 1, R³³ is 6-methoxycarbonyl and R³⁴ is hydrogen, and the combination of R¹ and R² for one compound corresponds in each case to one row of Table III

### Table 48

Compounds of the formula III in which n is 1, R³³ is 7-methoxycarbonyl and R³⁴ is hydrogen, and the combination of R¹ and R² for one compound corresponds in each case to one row of Table III

### Table 49

Compounds of the formula IIII in which n is 2, R³³ is 5,6-difluoro and R³⁴ is hydrogen, and the combination of R³¹ and R³² for one compound corresponds in each case to one row of Table III

### Table 50

Compounds of the formula III in which n is 2, R³³ is 5,6-dichloro and R³⁴ is hydrogen, and the combination of R³¹ and R³² for one compound corresponds in each case to one row of Table III

### Table 51

Compounds of the formula III in which n is 2, R³³ is 5,6-dibromo and R³⁴ is hydrogen, and the combination of R³¹ and R³² for one compound corresponds in each case to one row of Table III

### Table 52

Compounds of the formula III in which n is 2, R³³ is 5,6-difluoro and R³⁴ is fluorine, and the combination of R³¹ and R³² for one compound corresponds in each case to one row of Table III

### Table 53

Compounds of the formula III in which n is 2, R³³ is 5,6-dichloro and R³⁴ is fluorine, and the combination of R³¹ and R³² for one compound corresponds in each case to one row of Table III

### Table 54

Compounds of the formula III in which n is 2, R³³ is 5,6-dibromo and R³⁴ is fluorine, and the combination of R³¹ and R³² for one compound corresponds in each case to one row of Table III

### Table 55

Compounds of the formula III in which n is 2, R³³ is 5,6-difluoro and R³⁴ is chlorine, and the combination of R³¹ and R³² for one compound corresponds in each case to one row of Table III

### Table 56

Compounds of the formula III in which n is 2, R³³ is 5,6-dichloro and R³⁴ is chlorine, and the combination of R³¹ and R³² for one compound corresponds in each case to one row of Table III

### Table 57

Compounds of the formula III in which n is 2, R³³ is 5,6-dibromo and R³⁴ is chlorine, and the combination of R³¹ and R³² for one compound corresponds in each case to one row of Table III

### Table 58

Compounds of the formula III in which n is 2, R³³ is 5,6-difluoro and R³⁴ is methyl, and the combination of R³¹ and R³² for one compound corresponds in each case to one row of Table III

### Table 59

Compounds of the formula III in which n is 2, R³³ is 5,6-dichloro and R³⁴ is methyl, and the combination of R³¹ and R³² for one compound corresponds in each case to one row of Table III

### Table 60

Compounds of the formula III in which n is 2, R³³ is 5,6-dibromo and R³⁴ is methyl, and the combination of R³¹ and R³² for one compound corresponds in each case to one row of Table III

TABLE III

| No. | R³¹ | R³² |
|---|---|---|
| III-1 | H | H |
| III-2 | CH₃ | H |
| III-3 | CH₂CH₃ | H |
| III-4 | CH₂CH₂CH₃ | H |
| III-5 | F | H |
| III-6 | Cl | H |
| III-7 | Br | H |
| III-8 | I | H |
| III-9 | SCH₃ | H |
| III-10 | SCH₂CH₃ | H |
| III-11 | CF₃ | H |
| III-12 | CF₂CF₃ | H |
| III-13 | CN | H |
| III-14 | CHO | H |
| III-15 | COOCH₃ | H |
| III-16 | COOCH₂CH₃ | H |
| III-17 | C₆H₅ | H |
| III-18 | CH═NOH | H |
| III-19 | CH═NOCH₃ | H |
| III-20 | CH═NOC(═O)CH₃ | H |
| III-21 | H | Cl |
| III-22 | CH₃ | Cl |
| III-23 | CH₂CH₃ | Cl |
| III-24 | CH₂CH₂CH₃ | Cl |
| III-25 | F | Cl |
| III-26 | Cl | Cl |
| III-27 | Br | Cl |
| III-28 | I | Cl |
| III-29 | SCH₃ | Cl |
| III-30 | SCH₂CH₃ | Cl |
| III-31 | CF₃ | Cl |
| III-32 | CF₂CF₃ | Cl |
| III-33 | CN | Cl |
| III-34 | CHO | Cl |
| III-35 | COOCH₃ | Cl |
| III-36 | COOCH₂CH₃ | Cl |
| III-37 | C₆H₅ | Cl |
| III-38 | CH═NOH | Cl |
| III-39 | CH═NOCH₃ | Cl |
| III-40 | CH═NOC(═O)CH₃ | Cl |
| III-41 | H | F |
| III-42 | CH₃ | F |
| III-43 | CH₂CH₃ | F |
| III-44 | CH₂CH₂CH₃ | F |
| III-45 | F | F |
| III-46 | Cl | F |
| III-47 | Br | F |
| III-48 | I | F |
| III-49 | SCH₃ | F |
| III-50 | SCH₂CH₃ | F |
| III-51 | CF₃ | F |
| III-52 | CF₂CF₃ | F |
| III-53 | CN | F |
| III-54 | CHO | F |
| III-55 | COOCH₃ | F |
| III-56 | COOCH₂CH₃ | F |
| III-57 | C₆H₅ | F |
| III-58 | CH═NOH | F |
| III-59 | CH═NOCH₃ | F |
| III-60 | CH═NOC(═O)CH₃ | F |
| III-61 | H | Br |
| III-62 | CH₃ | Br |
| III-63 | CH₂CH₃ | Br |
| III-64 | CH₂CH₂CH₃ | Br |
| III-65 | F | Br |
| III-66 | Cl | Br |
| III-67 | Br | Br |
| III-68 | I | Br |
| III-69 | SCH₃ | Br |
| III-70 | SCH₂CH₃ | Br |
| III-71 | CF₃ | Br |
| III-72 | CF₂CF₃ | Br |
| III-73 | CN | Br |
| III-74 | CHO | Br |
| III-75 | COOCH₃ | Br |
| III-76 | COOCH₂CH₃ | Br |
| III-77 | C₆H₅ | Br |
| III-78 | CH═NOH | Br |
| III-79 | CH═NOCH₃ | Br |
| III-80 | CH═NOC(═O)CH₃ | Br |
| III-81 | H | I |
| III-82 | CH₃ | I |
| III-83 | CH₂CH₃ | I |
| III-84 | CH₂CH₂CH₃ | I |
| III-85 | F | I |
| III-86 | Cl | I |
| III-87 | Br | I |
| III-88 | I | I |
| III-89 | SCH₃ | I |
| III-90 | SCH₂CH₃ | I |
| III-91 | CF₃ | I |
| III-92 | CF₂CF₃ | I |
| III-93 | CN | I |
| III-94 | CHO | I |
| III-95 | COOCH₃ | I |
| III-96 | COOCH₂CH₃ | I |
| III-97 | C₆H₅ | I |
| III-98 | CH═NOH | I |
| III-99 | CH═NOCH₃ | I |
| III-100 | CH═NOC(═O)CH₃ | I |
| III-101 | H | CH₃ |
| III-102 | CH₃ | CH₃ |
| III-103 | CH₂CH₃ | CH₃ |
| III-104 | CH₂CH₂CH₃ | CH₃ |
| III-105 | F | CH₃ |
| III-106 | Cl | CH₃ |
| III-107 | Br | CH₃ |
| III-108 | I | CH₃ |
| III-109 | SCH₃ | CH₃ |
| III-110 | SCH₂CH₃ | CH₃ |
| III-111 | CF₃ | CH₃ |
| III-112 | CF₂CF₃ | CH₃ |
| III-113 | CN | CH₃ |
| III-114 | CHO | CH₃ |
| III-115 | COOCH₃ | CH₃ |
| III-116 | COOCH₂CH₃ | CH₃ |
| III-117 | C₆H₅ | CH₃ |
| III-118 | CH═NOH | CH₃ |
| III-119 | CH═NOCH₃ | CH₃ |
| III-120 | CH═NOC(═O)CH₃ | CH₃ |
| III-121 | H | CF₃ |
| III-122 | CH₃ | CF₃ |
| III-123 | CH₂CH₃ | CF₃ |
| III-124 | CH₂CH₂CH₃ | CF₃ |
| III-125 | F | CF₃ |
| III-126 | Cl | CF₃ |
| III-127 | Br | CF₃ |
| III-128 | I | CF₃ |
| III-129 | SCH₃ | CF₃ |
| III-130 | SCH₂CH₃ | CF₃ |
| III-131 | CF₃ | CF₃ |
| III-132 | CF₂CF₃ | CF₃ |
| III-133 | CN | CF₃ |
| III-134 | CHO | CF₃ |
| III-135 | H | C(═O)OCH₃ |
| III-136 | CH₃ | C(═O)OCH₃ |
| III-137 | CH₂CH₃ | C(═O)OCH₃ |
| III-138 | CH₂CH₂CH₃ | C(═O)OCH₃ |
| III-139 | F | C(═O)OCH₃ |
| III-140 | Cl | C(═O)OCH₃ |
| III-141 | Br | C(═O)OCH₃ |
| III-142 | I | C(═O)OCH₃ |
| III-143 | SCH₃ | C(═O)OCH₃ |
| III-144 | SCH₂CH₃ | C(═O)OCH₃ |
| III-145 | CF₃ | C(═O)OCH₃ |
| III-146 | CF₂CF₃ | C(═O)OCH₃ |
| III-147 | CN | C(═O)OCH₃ |
| III-148 | H | C(═O)OCH₂CH₃ |
| III-149 | CH₃ | C(═O)OCH₂CH₃ |
| III-150 | CH₂CH₃ | C(═O)OCH₂CH₃ |
| III-151 | CH₂CH₂CH₃ | C(═O)OCH₂CH₃ |
| III-152 | F | C(═O)OCH₂CH₃ |
| III-153 | Cl | C(═O)OCH₂CH₃ |
| III-154 | Br | C(═O)OCH₂CH₃ |
| III-155 | I | C(═O)OCH₂CH₃ |
| III-156 | SCH₃ | C(═O)OCH₂CH₃ |
| III-157 | SCH₂CH₃ | C(═O)OCH₂CH₃ |
| III-158 | CF₃ | C(═O)OCH₂CH₃ |
| III-159 | CF₂CF₃ | C(═O)OCH₂CH₃ |
| III-160 | CN | C(═O)OCH₂CH₃ |
| III-161 | H | C(═O)OCH₂CH₂CH₃ |
| III-162 | CH₃ | C(═O)OCH₂CH₂CH₃ |
| III-163 | CH₂CH₃ | C(═O)OCH₂CH₂CH₃ |
| III-164 | CH₂CH₂CH₃ | C(═O)OCH₂CH₂CH₃ |
| III-165 | F | C(═O)OCH₂CH₂CH₃ |
| III-166 | Cl | C(═O)OCH₂CH₂CH₃ |
| III-167 | Br | C(═O)OCH₂CH₂CH₃ |
| III-168 | I | C(═O)OCH₂CH₂CH₃ |
| III-169 | SCH₃ | C(═O)OCH₂CH₂CH₃ |
| III-170 | SCH₂CH₃ | C(═O)OCH₂CH₂CH₃ |
| III-171 | CF₃ | C(═O)OCH₂CH₂CH₃ |
| III-172 | CF₂CF₃ | C(═O)OCH₂CH₂CH₃ |
| III-173 | CN | C(═O)OCH₂CH₂CH₃ |
| III-174 | H | C(═O)OCH(CH₃)₂ |
| III-175 | CH₃ | C(═O)OCH(CH₃)₂ |
| III-176 | CH₂CH₃ | C(═O)OCH(CH₃)₂ |
| III-177 | CH₂CH₂CH₃ | C(═O)OCH(CH₃)₂ |
| III-178 | F | C(═O)OCH(CH₃)₂ |
| III-179 | Cl | C(═O)OCH(CH₃)₂ |
| III-180 | Br | C(═O)OCH(CH₃)₂ |
| III-181 | I | C(═O)OCH(CH₃)₂ |
| III-182 | SCH₃ | C(═O)OCH(CH₃)₂ |
| III-183 | SCH₂CH₃ | C(═O)OCH(CH₃)₂ |
| III-184 | CF₃ | C(═O)OCH(CH₃)₂ |
| III-185 | CF₂CF₃ | C(═O)OCH(CH₃)₂ |
| III-186 | CN | C(═O)OCH(CH₃)₂ |
| III-187 | H | C(═O)OCH₂CH₂CH₂CH₃ |
| III-188 | CH₃ | C(═O)OCH₂CH₂CH₂CH₃ |
| III-189 | CH₂CH₃ | C(═O)OCH₂CH₂CH₂CH₃ |
| III-190 | CH₂CH₂CH₃ | C(═O)OCH₂CH₂CH₂CH₃ |
| III-191 | F | C(═O)OCH₂CH₂CH₂CH₃ |
| III-192 | Cl | C(═O)OCH₂CH₂CH₂CH₃ |
| III-193 | Br | C(═O)OCH₂CH₂CH₂CH₃ |
| III-194 | I | C(═O)OCH₂CH₂CH₂CH₃ |
| III-195 | SCH₃ | C(═O)OCH₂CH₂CH₂CH₃ |
| III-196 | SCH₂CH₃ | C(═O)OCH₂CH₂CH₂CH₃ |
| III-197 | CF₃ | C(═O)OCH₂CH₂CH₂CH₃ |
| III-198 | CF₂CF₃ | C(═O)OCH₂CH₂CH₂CH₃ |
| III-199 | CN | C(═O)OCH₂CH₂CH₂CH₃ |

Particular preference is given to the combinations of a compound I with one of the compounds III-135, III-161 and III-187 of Table 3, III-27 of Table 4, III-62 of Table 7 and III-22 of Table 55.

In another embodiment of the invention, mixtures of a compound I with at least one compound of the formula IV are present.

In formula IV, the following meanings of the substituents are preferred, alone or in combination:

Ar is preferably phenyl or a five-membered aromatic heterocycle, in particular a five-membered heteroaryl radical which is unsubstituted or substituted by one or two groups R⁴¹.

In addition, Ar preferably denotes the following groups: phenyl, pyridine, pyrazine, furan, thiophene, pyrazole and thiazole. Particularly preferred groups Ar are: 3-pyridinyl, pyrazinyl, 3-furyl, 3-thiophenyl, 4-pyrazolyl, 5-thiazolyl.

With particular preference, a group R⁴¹ is located in the ortho-position to the amide grouping.

Preferred groups R⁴¹ are halogen, in particular chlorine, alkyl, in particular methyl, and halomethyl, in particular fluoromethyl, difluoromethyl or trifluoromethyl.

Preferred groups R are alkyl groups, in particular branched C₃-C₈-alkyl groups, in particular 4-methylpent-2-yl.

For the intended use in a mixture with the compound I, the following compounds of the formula IV are particularly suitable:

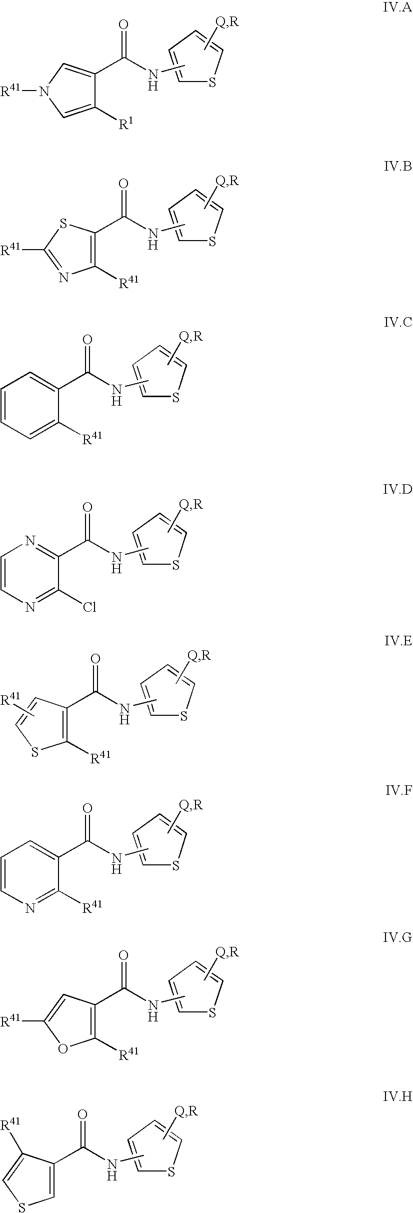

If two groups R⁴¹ are present in one formula, these groups can be identical or different.

Particularly preferred are compounds IV.A, in particular compounds of the formulae IV.A.1 and IV.B.1 in which the radicals R⁴¹ can be identical or different and are methyl and halomethyl and R is alkyl, such as branched C₃-C₈-alkyl, in particular 4-methyl-pent-2-yl:

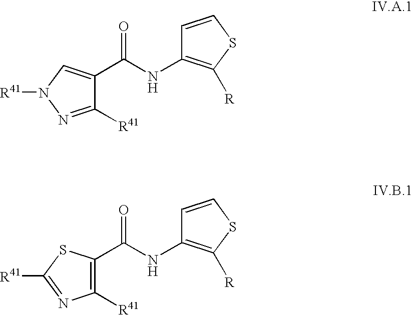

Especially preferred are compounds IV.A.11 (common name: penthiopyrad) and IV.B.11 which are present in the form of their R and S isomers:

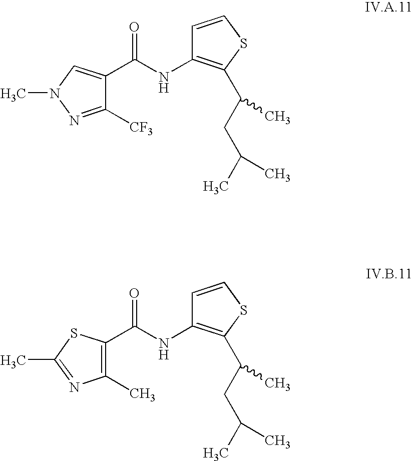

In a further embodiment of the invention, mixtures of a compound I with at least one oxime ether derivative of the formula V are present.

Among the compounds of the formula V, preference is given to those in which X is a difluoromethoxy group. In addition, particular preference is given to compounds of the formula V in which the index n is zero.

Particularly preferred compounds V are in particular the compounds listed in Table V below:

TABLE V

| No. | X | Rₙ |
|---|---|---|
| V-1 | OCF₃ | H |
| V-2 | OCHF₂ | H |
| V-3 | OCH₂F | H |
| V-4 | OCF₃ | 4-OCH₃ |
| V-5 | OCHF₂ | 4-OCH₃ |
| V-6 | OCH₂F | 4-OCH₃ |
| V-7 | OCF₃ | 4-F |
| V-8 | OCHF₂ | 4-F |
| V-9 | OCH₂F | 4-F |
| V-10 | OCF₃ | 4-Cl |
| V-11 | OCHF₂ | 4-Cl |
| V-12 | OCH₂F | 4-Cl |
| V-13 | OCF₃ | 4-CH₃ |
| V-14 | OCHF₂ | 4-CH₃ |
| V-15 | OCH₂F | 4-CH₃ |
| V-16 | OCF₃ | 4-CF₃ |
| V-17 | OCHF₂ | 4-CF₃ |
| V-18 | OCH₂F | 4-CF₃ |
| V-19 | OCF₃ | 4-CF₃ |

Especially preferred is the compound V-2.

In a further embodiment of the invention, mixtures of a compound I with at least one compound of the formula VI are present.

With a view to the intended use of the compounds VI, particular preference is given to the following meanings of the substituents, in each case alone or in combination:

R⁶¹ is hydrogen;

R⁶² is C₁-C₆-alkyl; such as methyl and ethyl, in particular methyl,

R⁶³ is C₁-C₆-alkyl, such as methyl and ethyl, in particular ethyl;

R⁶⁴ is C₁-C₆-alkyl, in particular methyl;

R⁶⁵ is C₁-C₆-alkyl, in particular methyl;

m is 1, where R⁶⁵ is located in the para-position to R⁴;

A is oxygen (—O—);

R⁶⁶ is phenyl which is preferably unsubstituted or substituted by one to three groups R^{f}, in particular by one or two groups R^{f};

R^{f} is halogen, in particular fluorine or chlorine, alkyl, in particular methyl, ethyl, n- and isopropyl and tert-butyl, and haloalkyl, in particular trifluoromethyl.

The groups R^{f} are preferably located in the 3- or 3,4-position.

Suitable for the intended use in mixtures with the compound I are in particular the compounds of the formula VI.A listed in Table VI below:

TABLE VI

| | |
|---|---|
| | VI.A |
| 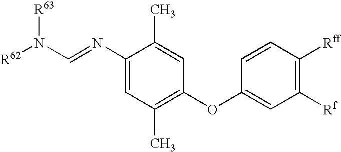 | |

| No. | R⁶² | R⁶³ | R^{f} | R^{ff} |
|---|---|---|---|---|
| VI-1 | CH₃ | CH₂CH₃ | CF₃ | Cl |
| VI-2 | CH₃ | CH₂CH₃ | CF₃ | F |
| VI-3 | CH₃ | CH₃ | CF₃ | H |
| VI-4 | CH₂CH₃ | CH₂CH₃ | CF₃ | H |
| VI-5 | CH₃ | CH₃ | C(CH₃)₃ | H |
| VI-6 | CH₂CH₃ | CH₂CH₃ | C(CH₃)₃ | H |
| VI-7 | CH₃ | CH₃ | C₆H₅—O— | H |
| VI-8 | CH₂CH₃ | CH₂CH₃ | C₆H₅—O— | H |
| VI-9 | CH₃ | CH₃ | Cl | Cl |
| VI-10 | CH₂CH₃ | CH₂CH₃ | Cl | Cl |

In another embodiment of the invention, mixtures of a compound I with at least one compound of the formula VII are present.

Preference is given to compounds of the formula VII in which R⁷¹ is n-propyl or n-butyl, in particular n-propyl.

In addition, particular preference is given to compounds of the formula VII in which R⁷³ is iodine or bromine, in particular iodine.

The formula VII represents in particular compounds of the formulae VII.1, VII.2 and VII.3:

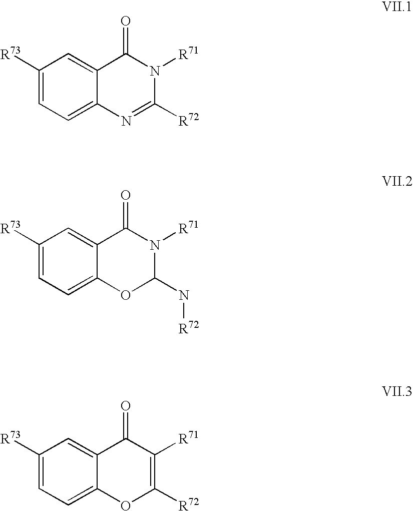

in which the variables are as defined for formula VIII.

The compound of the formula VII.1 in which R⁷¹ is n-propyl, R⁷² is n-propoxy and R⁷³ is iodine is also known under the common name proquinazid (compound VII. 1-1). Mixtures of a compound of the formula I and proquinazid are a preferred embodiment of the invention.

In addition, mixtures comprising a compound of the formula I and a compound of the formula VII.2 are also a preferred embodiment of the invention.

Especially preferred are mixtures of a compound I and one of the following compounds of the formula VII.2:

| No. | R⁷¹ | R⁷² | R⁷³ |
|---|---|---|---|
| VII.2-1 | CH₂CH₂CH₃ | OCH₃ | I |
| VII.2-2 | CH₂CH₂CH₂CH₃ | OCH₂CH₃ | I |
| VII.2-3 | CH₂CH₂CH₃ | OCH₂CH₃ | I |
| VII.2-4 | CH₂CH₂CH₃ | OCH(CH₃)₂ | I |

A further preferred embodiment of the invention relates to mixtures of a compound I and one of the compounds of the formula VII.3 below:

| No. | R¹ | R² | R³ |
|---|---|---|---|
| VII.3-1 | CH₂CH₂CH₃ | OCH₃ | I |
| VII.3-2 | CH₂CH₂CH₂CH₃ | OCH₂CH₃ | I |
| VII.3-3 | CH₂CH₂CH₃ | OCH₂CH₃ | I |
| VII.3-4 | CH₂CH₂CH₃ | OCH(CH₃)₂ | I |
| VII.3-5 | CH₂CH₂CH₃ | OCH₂CH₂CH₃ | I |
| VII.3-6 | CH₂CH₂CH₃ | OCH₂CH₂CH₂CH₃ | I |
| VII.3-7 | CH₂CH₂CH₃ | OCH₃ | Br |
| VII.3-8 | CH₂CH₂CH₂CH₃ | OCH₂CH₃ | Br |
| VII.3-9 | CH₂CH₂CH₃ | OCH₂CH₃ | Br |
| VII.3-10 | CH₂CH₂CH₃ | OCH(CH₃)₂ | Br |
| VII.3-11 | CH₂CH₂CH₃ | OCH₂CH₂CH₃ | Br |
| VII.3-12 | CH₂CH₂CH₃ | OCH₂CH₂CH₂CH₃ | Br |

Preferred mixing partners for the compounds I are active compounds from groups A) to R) selected from one of the following groups:
A) azoles, such as bromoconazole, cyproconazole, difenoconazole, dinitroconazole, epoxiconazole, fenbuconazole, fluquinconazole, flusilazole, flutriafol, hexaconazole, ipconazole, metconazole, myclobutanil, penconazole, propiconazole, prochloraz, prothioconazole, simeconazole, tebuconazole, tetraconazole, triflumizol, triticonazole;
B) strobilurins, such as azoxystrobin, dimoxystrobin, fluoxastrobin, kresoxim-methyl, orysastrobin, picoxystrobin, pyraclostrobin or trifloxystrobin;
C) acylalanines, such as metalaxyl, mefenoxam;
D) amine derivatives, such as dodine, fenpropimorph, fenpropidin, spiroxamin,
tridemorph;
E) anilinopyrimidines, such as pyrimethanil, mepanipyrim or cyprodinil;
F) dicarboximides, such as iprodione, procymidon, vinclozolin;
G) cinnamides and analogs, such as dimethomorph or flumorph;
K) dithiocarbamates, such as maneb, mancozeb, metiram, thiram;
L) heterocyclic compounds, selected from the group consisting of benomyl, boscalid, carbendazim, carboxin, cyazofamid, dithianon, fenarimol, flutolanil, penthiopyrad, picobenzamid, proquinazid, pyrifenox, quinoxyfen, thiophanate-methyl;
   benzimidazole derivatives of the formula II;
   sulfamoyl compounds of the formula III;
M) sulfur and copper fungicides, such as Bordeaux mixtures, copper acetate, copper oxychloride, basic copper sulfate;
N) nitrophenyl derivatives, such as dinocap;
J) phenylpyrroles, such as fenpiclonil or fludioxonil;
P) sulfenic acid derivatives, such as captan, folpet, tolylfluanid;
Q) other fungicides, such as benthiavalicarb, chlorothalonil, cyflufenamid, diclofluanid, fenhexamid, fluazinam, fosetyl, fosetyl-aluminum, phosphorous acid, iprovalicarb, metrafenone, pencycuron;
   oxime ether derivatives of the formula V,
   phenylamidine derivatives of the formula VI,
   compounds of the formula VII, and
R) growth retardants, such as prohexadione calcium, trinexapac-ethyl, chlormequat, mepiquat-chloride and diflufenzopyr.

Owing to the basic character of their nitrogen atoms or carboxylate groups, the compounds I and the active compounds from groups A) to R) are capable of forming salts or adducts with inorganic or organic acids or with metal ions.

Examples of inorganic acids are hydrohalic acids, such as hydrogen fluoride, hydrogen chloride, hydrogen bromide and hydrogen iodide, sulfuric acid, phosphoric acid and nitric acid.

Suitable organic acids are, for example, formic acid, carbonic acid, and alkanoic acids, such as acetic acid, trifluoroacetic acid, trichloroacetic acid and propionic acid, and also glycolic acid, lactic acid, succinic acid, citric acid, benzoic acid, cinnamic acid, oxalic acid, p-toluenesulfonic acid, salicylic acid, p-aminosalicylic acid, 2-phenoxybenzoic acid and 2-acetoxybenzoic acid.

Suitable metal ions are in particular the ions of the elements of transition groups one to eight, in particular chromium, manganese, iron, cobalt, nickel, copper, zinc, of the first main group, in particular lithium, sodium and potassium, and of the second main group, in particular calcium and magnesium, and of the third and fourth main groups, in particular aluminum, tin and lead. If appropriate, the metal ions can be present in the various valencies that they can assume.

When preparing the mixtures, it is preferred to employ the pure active compounds I and II, to which further active compounds against harmful fungi or against other pests, such as insects, arachnids or nematodes, or else herbicidal or growth-regulating active compounds or fertilizers, can be added according to need.

Suitable “further active compounds” in the above sense are in particular fungicides from groups A) to R) defined at the outset, in particular the preferred representatives mentioned above.

In addition to a compound of the formula I and an active compound from groups A) to R), the mixtures according to the invention may comprise, as active components, further active components from compounds of the formula I and active compounds from groups A) to R).

One embodiment of the mixtures comprises, in addition to a compound of the formula I and an active compound from groups A) to R), as active components, one or two, in particular one active compound from the groups A) to R).

The compound I and the active compound from groups A) to R) are usually applied in a weight ratio of from 100:1 to 1:100, preferably from 20:1 to 1:20, in particular from 10:1 to 1:10. In the case of mixtures of a compound I and diflufenzopyr, mixing ratios of from 1000:1 to 1:1 are also possible.

The further active components are, if desired, added in a ratio of from 20:1 to 1:20 to the compound I.

Depending on the type of compound and the desired effect, the application rates of the mixtures according to the invention are from 5 g/ha to 2000 g/ha, preferably from 50 to 900 g/ha, in particular from 50 to 750 g/ha.

Correspondingly, the application rates for the compound I are generally from 1 to 1000 g/ha, preferably from 10 to 900 g/ha, in particular from 20 to 750 g/ha.

Correspondingly, the application rates for the active compound from groups A) to R) are generally from 1 to 2000 g/ha, preferably from 10 to 900 g/ha, in particular from 40 to 750 g/ha. The application rates for diflufenzopyr are usually from 0.01 to 50 g/ha, preferably from 0.1 to 10 g/ha.

In the treatment of seed, application rates of mixture are generally from 1 to 1000 g/100 kg of seed, preferably from 1 to 750 g/100 kg, in particular from 5 to 500 g/100 kg.

The method for controlling harmful fungi is carried out by the separate or joint application of the compound I and the active.compound from groups A) to R) or of the mixtures of the compound I and the active compound from groups A) to R) by spraying or dusting the seeds, the plants or the soil before or after sowing of the plants or before or after emergence of the plants.

The mixtures according to the invention, or the active components, can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner, for example by extending the active compound with solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

Suitable for use as surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compounds. The active compounds are employed in a purity of from 90% to 100%, preferably 95% to 100% (according to NMR spectrum).

The following are examples of formulations of the invention:

1. Products for Dilution with Water

### A Water-soluble Concentrates (SL)

10 parts by weight of the active compounds are dissolved with 90 parts by weight of water or a water-soluble solvent. As an alternative, wetters or other auxiliaries are added. The active compound dissolves upon dilution with water. In this way, a formulation having an active compound content of 10% by weight is obtained.

### B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

### D Emulsions (EW, EO)

25 parts by weight of the active compounds are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (for example Ultraturrax) and made into a homogeneous emulsion. Dilution with water gives an emulsion. The formulation has an active compound content of 25% by weight.

### E Suspensions (SC, OD)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

### F Water-Dispersible Granules and Water-Soluble Granules (WG, SG)

50 parts by weight of the active compounds are ground finely with addition of 50 parts by weight of dispersants and wetters and prepared as water-dispersible or water-soluble granules by means of technical appliances (for example extrusion, spray tower, fluidized bed). Dilution with water gives a stable dispersion or solution of the active compound. The formulation has an active compound content of 50% by weight.

### G Water-Dispersible Powders and Water-Soluble Powders (WP, SP)

75 parts by weight of the active compounds are ground in a rotor-stator mill with addition of 25 parts by weight of dispersants, wetters and silica gel. Dilution with water gives a stable dispersion or solution of the active compound. The active compound content of the formulation is 75% by weight.

### 2. Products to be Applied Undiluted

### H Dustable Powders (DP)

5 parts by weight of the active compounds are ground finely and mixed intimately with 95 parts by weight of finely divided kaolin. This gives a dustable product having an active compound content of 5% by weight.

### I Granules (GR, FG, GG, MG)

0.5 part by weight of the active compounds is ground finely and associated with 99.5 parts by weight of carriers. Current methods are extrusion, spray-drying or the fluidized bed. This gives granules to be applied undiluted having an active compound content of 0.5% by weight.

### J ULV Solutions (UL)

10 parts by weight of the active compounds are dissolved in 90 parts by weight of an organic solvent, for example xylene. This gives a product to be applied undiluted having an active compound content of 10% by weight.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), it being possible to apply formulations comprising over 95% by weight of active compound, or even to apply the active compound without additives.

Oils of various types, wetters, adjuvants, herbicides, fungicides, other pesticides, or bactericides may be added to the active compounds even, if appropriate, not until immediately prior to use (tank mix). These agents are typically admixed with the compositions according to the invention in a weight ratio of from 1:10 to 10:1.

The compounds I and II or the mixtures or the corresponding formulations are applied by treating the harmful fungi, the plants, seeds, soils, areas, materials or spaces to be kept free from them with a fungicidally effective amount of the mixture or, in the case of separate application, of the compounds I and II. Application can be carried out before or after infection by the harmful fungi.

### SYNTHESIS EXAMPLES

The novel compounds of the formula I can be prepared analogously to the process known from U.S. Pat. No. 5,593,996. With appropriate modification of the starting materials, the procedure given in the synthesis example below was used to obtain further compounds I. The compounds obtained in this manner are listed in the table below, together with physical data.

### Example 1

### Preparation of 5-chloro-6-(2-methylphenyl)-7-(4-methylpiperidin-1-yl)-1,2,4-triazolo[1,5a]pyrimidine[I-9]

A solution of 1 g (3.6 mmol) of 5,7-dichloro-6-(2-methylphenyl)-1,2,4-tri-azolo[1,5a]pyrimidine (prepared analogously to WO 1994/20501), 0.36 g (3.62 mmol) of 4-methylpiperidine and 0.37 g (3.62 mmol) of triethylamine in 10 ml of methylene chloride was stirred at about 20-25° C. for about 1.5 hours. The reaction mixture was then freed from the solvent and the residue was taken up in acetonitrile. Using preparative MPLC on silica gel RP-18, this yielded 1.1 g of the title compound as a colorless crystalline material of m.p. 170-180° C.

¹H-NMR (CDCl₃, δ in ppm): 8.4 (s, 1H); 7.3 (m, 3H); 7.1 (d, 1H); 3.85 (m, 1H); 3.45 (m, 1H); 2.7 (m, 1H); 2.6 (m, 1H); 2.2 (s, 3H); 1.55 (m, 2H); 1.45 (m, 1H); 1.25 (m, 2H); 0.9 (d, 3H)

TABLE I
Compounds of the formula I

| | | | Physical data |
|---|---|---|---|
| No. | D | L | (m.p. [° C.]) |
| I-1 | —(CH₂)₅— | CH₃ | 167-168 |
| I-2 | —CH₂—CH(CH₃)—(CH₂)₃— | F | 133-134 |
| I-3 | —(CH₂)₂—CH(CH[CH₃]₂)—CH₂)₂— | Cl | 167 |
| I-4 | —(CH₂)₂—CH(CH[CH₃]₂)—CH₂)₂— | F | 166 |
| I-5 | —CH(CH₃)—CH₂—CH(CH₃)—(CH₂)₂— | F | 169-172 |
| I-6 | —CH(CH₃)—CH₂—CH(CH₃)—(CH₂)₂— | Cl | 172-174 |
| I-7 | —CH(CH₃)—(CH₂)₄— | CH₃ | 135 |
| I-8 | —(CH₂)₂—CH(CH[CH₃]₂)—CH₂)₂— | CH₃ | 65-70 |
| I-9 | —(CH₂)₂—CH(CH₃)—(CH₂)₂— | CH₃ | 170-180 |
| I-10 | —(CH₂)₂—CH(CH₃)—(CH₂)₂— | F | 210-212 |
| I-11 | —(CH₂)₄— | F | 171-173 |
| I-12 | —CH₂—CH(CH₃)—(CH₂)₂— | F | 154-156 |

The fungicidal effect of the compound and the mixtures was demonstrated by the following tests:

### Use Example 1

### Activity Against Venturia Inequalis (Protective)

Leaves of potted apple seedlings of the cultivar “Common” were sprayed to run off point with an aqueous active compound preparation which had been prepared using a stock solution of 5% active compound, 94% acetonitrile and 1% emulsifier (Tween 20). After the spray coating had dried on (3-5 h), the leaves were inoculated with an aqueous spore suspension of Venturia inequalis. The test plants were then placed in climatized chambers at 22-24° C. and 95-99% relative atmospheric humidity for 2 days and then cultivated in the greenhouse at 21-23° C. and about 95% relative atmospheric humidity for a further 2 weeks. The extent of the development of the infection on the leaves was then determined visually.

In this test, the plants which had been treated with 200 ppm of the active compound I-1, I-2 or I-10 showed an infection of at most 15%, whereas the untreated plants were 90% infected.

### Use Example 2

### Activity Against Alternaria solani on Tomatoes (Protective)

Leaves of tomato plants of the cultivar “Pixie II” which had been cultivated in pots up to the 4-leaf stage were sprayed to run off point with an aqueous active compound preparation which had been prepared from a stock solution of 5% active compound, 94% acetone and 1% emulsifier (Tween 20). After the spray coating had dried on (3-5 h), the leaves were inoculated with an aqueous spore suspension of Alternaria solani (density 15×10³ spores per ml). The test plants were then placed in climatized chambers at 22-24° C. and 96-99% relative atmospheric humidity for 36 hours and then cultivated in the greenhouse at 21-23° C. and about 95% relative atmospheric humidity for a further 2 to 3 days. The extent of the development of the infection on the leaves was then determined visually.

In this test, plants which had been treated with 200 ppm of the active compounds I-1, I-2, I-5, I-6, I-7, I-9 or I-11 showed an infection of at most 15%, whereas the untreated plants were 90% infected.

For use examples 3 to 9, the active compounds, separately or jointly, were prepared as a stock solution comprising 0.25% by weight of active compound in acetone or DMSO. 1% by weight of the emulsifier Uniperol® EL (wetter having emulsifying and dispersant action based on ethoxylated alkylphenols) was added to this solution, and the mixture was diluted with water to the desired concentration.

The active compounds fosetyl-AI, epoxiconazole, triticonazole and pyraclostrobin were applied in their commercial formulation.

### Use Example 3

### Activity Against Early Blight of Tomato Caused by Alternaria Solani

Leaves of potted plants of the cultivar “Goldene Königin” were sprayed to run off point with an aqueous suspension having the active compound concentration stated below. The next day, the leaves were infected with an aqueous spore suspension of Alternaria solani in a 2% strength biomalt solution having a density of 0.17×10⁶ spores/ml. The plants were then placed in a water vapor-saturated chamber at temperatures between 20 and 22° C. After 5 days, the disease on the untreated but infected control plants had developed to such an extent that the infection could be determined visually in %.

In this test, the plants which had been treated with 63 ppm of the active compound I-12 showed no infection, whereas the untreated plants were 90% infected.

### Use Example 4

### Activity Against Net Blotch of Barley Caused by Pyrenophora Teres, 1 Day Protective Application

Leaves of potted barley seedlings were sprayed to run off with an aqueous suspension having the active compound concentration stated below. 24 hours after the spray coating had dried on, the test plants were inoculated with an aqueous spore suspension of Pyrenophora [syn. Drechslera ] teres, the net blotch pathogen. The test plants were then placed in a greenhouse at temperatures between 20 and 24° C. and 95 to 100% relative atmospheric humidity. After 6 days, the extent of the development of the disease was determined visually in % infection of the entire leaf area.

In this test, the plants which had been treated with 63 ppm of the active compound I-12 showed an infection of 7%, whereas the untreated plants were 90% infected.

### Synergistic Effects

### Use Example 5

### Activity Against Early Blight of Tomato Caused by Alternaria Solani

A further test according to use example 3 was evaluated as follows:

The visually determined percentages of infected leaf areas were converted into efficacies in % of the untreated control:

The efficacy (E) is calculated as follows using Abbot's formula:

E=(1−α/β)·100

α corresponds to the fungicidal infection of the treated plants in % and
β corresponds to the fungicidal infection of the untreated (control) plants in %

An efficacy of 0 means that the infection level of the treated plants corresponds to that of the untreated control plants; an efficacy of 100 means that the treated plants were not infected.

The expected efficacies of mixtures of active compounds were determined using Colby's formula (Colby, S.R. “Calculating synergistic and antagonistic responses of herbicide combinations”, Weeds, 15, 20-22, 1967) and compared with the observed efficacies.

Colby's formula:

E=x+y−x·y/100
E expected efficacy, expressed in % of the untreated control, when using the mixture of the active compounds A and B at the concentrations a and b
x efficacy, expressed in % of the untreated control, when using the active compound A at the concentration a
y efficacy, expressed in % of the untreated control, when using the active compound B at the concentration b

The following results were obtained:

TABLE 1
Individual active compounds

| | | Active compound | Efficacy in % |
|---|---|---|---|
| | | concentration in | of the untreated |
| Example | Active compound | the spray liquor [ppm] | control |
| 1 | Control (untreated) | — | (90% infection) |
| 2 | A-8 | 16 | 33 |
| | | 4 | 0 |
| 3 | A-27 | 63 | 67 |
| | | 16 | 22 |
| | | 4 | 11 |
| 4 | A-31 | 63 | 78 |
| 5 | epoxiconazole | 4 | 56 |
| 6 | prothioconazole | 4 | 22 |
| 7 | triticonazole | 4 | 0 |
| 8 | mancozeb | 63 | 0 |
| | | 16 | 0 |
| 9 | cyazofamid | 250 | 0 |
| | | 63 | 0 |
| | | 16 | 0 |
| 10 | folpet | 63 | 0 |
| 11 | fosethyl-Al | 250 | 0 |
| 12 | chlorothalonil | 63 | 0 |
| 13 | boscalid | 16 | 22 |
| 14 | metalaxyl | 4 | 0 |

TABLE 2
Mixtures according to the invention

| | Active compound mixture | | |
|---|---|---|---|
| | Concentration | | Calculated |
| Example | Mixing ratio | Observed efficacy | efficacy*) |
| 15 | A-8 + epoxiconazole | 94 | 70 |
| | 16 + 4 ppm | | |
| | 4:1 | | |
| 16 | A-8 + epoxiconazole | 67 | 56 |
| | 4 + 4 ppm | | |
| | 1:1 | | |
| 17 | A-27 + epoxiconazole | 89 | 65 |
| | 16 + 4 ppm | | |
| | 4:1 | | |
| 18 | A-27 + epoxiconazole | 78 | 60 |
| | 4 + 4 ppm | | |
| | 1:1 | | |
| 19 | A-27 + prothioconazole | 100 | 74 |
| | 63 + 4 ppm | | |
| | 16:1 | | |
| 20 | A-27 + triticonazole | 100 | 67 |
| | 63 + 4 ppm | | |
| | 16:1 | | |
| 21 | A-31 + mancozeb | 100 | 78 |
| | 63 + 63 ppm | | |
| | 1:1 | | |
| 22 | A-31 + mancozeb | 92 | 78 |
| | 63 + 16 ppm | | |
| | 4:1 | | |
| 23 | A-27 + cyazofamid | 83 | 67 |
| | 63 + 16 ppm | | |
| | 4:1 | | |
| 24 | A-31 + Cyazofamid | 100 | 78 |
| | 63 + 250 ppm | | |
| | 1:4 | | |
| 25 | A-31 + Cyazofamid | 100 | 78 |
| | 63 + 63 ppm | | |
| | 1:1 | | |
| 26 | A-31 + Cyazofamid | 94 | 78 |
| | 63 + 16 ppm | | |
| | 4:1 | | |
| 27 | A-27 + Folpet | 94 | 67 |
| | 63 + 250 ppm | | |
| | 1:4 | | |
| 28 | A-27 + fosethyl-Al | 89 | 67 |
| | 63 + 250 ppm | | |
| | 1:4 | | |
| 29 | A-31 + chlorothalonil | 89 | 78 |
| | 63 + 63 ppm | | |
| | 1:1 | | |
| 30 | A-8 + boscalid | 67 | 48 |
| | 16 + 16 ppm | | |
| | 1:1 | | |
| 31 | A-27 + metalaxyl | 67 | 22 |
| | 16 + 4 ppm | | |
| | 4:1 | | |

*)efficacy calculated using Colby's formula

### Use Example 6

### Protective Activity Against Puccinia Recondita on Wheat (Brown Rust of Wheat)

Leaves of potted wheat seedlings of the cultivar “Kanzler” were sprayed to run off point with an aqueous suspension having the active compound concentration stated below. The next day, the treated plants were inoculated with a spore suspension of brown rust of wheat (Puccinia recondite). The plants were then placed in a chamber with high atmospheric humidity (90 to 95%) at 20 to 22° C. for 24 hours. During this time, the spores germinated and the germ tubes penetrated into the leaf tissue. The next day, the test plants were returned to the greenhouse and cultivated at temperatures between 20 and 22° C. and 65 to 70% relative atmospheric humidity for a further 7 days. The extent of the rust fungus development on the leaves was then determined visually.

The following results were obtained:

TABLE 3
Individual active compounds

| | | Active compound | Efficacy in % of |
|---|---|---|---|
| | | concentration in | the untreated |
| Example | Active compound | the spray liquor [ppm] | control |
| 32 | Control | — | (90% infection) |
| | (untreated) | | |
| 33 | A-8 | 63 | 67 |
| | | 16 | 33 |
| 34 | A-27 | 63 | 50 |
| | | 16 | 50 |
| | | 4 | 33 |
| 35 | A-31 | 16 | 22 |
| 36 | kresoxim-methyl | 4 | 0 |
| | | 1 | 0 |
| 37 | mancozeb | 63 | 0 |
| | | 16 | 0 |
| 38 | fosethyl-Al | 63 | 0 |
| 39 | chlorothalonil | 16 | 17 |
| 40 | fenpropimorph | 63 | 0 |
| | | 16 | 0 |
| 41 | dimethomorph | 63 | 0 |
| | | 16 | 0 |
| 41 | spiroxamine | 63 | 0 |
| | | 16 | 0 |
| 43 | prochloraz | 16 | 0 |
| | | 4 | 0 |
| 44 | pyrimethanil | 16 | 0 |

TABLE 4
Mixtures according to the invention

| | Active compound mixture | | |
|---|---|---|---|
| | Concentration | | Calculated |
| Example | Mixing ratio | Observed efficacy | efficacy*) |
| 45 | A-31 + kresoxim-methyl | 72 | 22 |
| | 16 + 4 ppm | | |
| | 4:1 | | |
| 46 | A-31 + kresoxim-methyl | 67 | 22 |
| | 16 + 1 ppm | | |
| | 16:1 | | |
| 47 | A-8 + mancozeb | 92 | 33 |
| | 16 + 63 ppm | | |
| | 1:4 | | |
| 48 | A-8 + mancozeb | 98 | 67 |
| | 63 + 16 ppm | | |
| | 4:1 | | |
| 49 | A-8 + fosethyl-Al | 75 | 33 |
| | 16 + 63 ppm | | |
| | 1:4 | | |
| 50 | A-8 + chlorothalonil | 88 | 44 |
| | 16 + 16 ppm | | |
| | 1:1 | | |
| 51 | A-8 + chlorothalonil | 95 | 72 |
| | 63 + 16 ppm | | |
| | 4:1 | | |
| 52 | A-8 + fenpropimorph | 83 | 67 |
| | 63 + 16 ppm | | |
| | 4:1 | | |
| 53 | A-8 + fenpropimorph | 67 | 33 |
| | 16 + 63 ppm | | |
| | 1:4 | | |
| 54 | A-27 + fenpropimorph | 92 | 50 |
| | 63 + 16 ppm | | |
| | 4:1 | | |
| 55 | A-27 + fenpropimorph | 92 | 50 |
| | 16 + 63 ppm | | |
| | 1:4 | | |
| 56 | A-8 + dimethomorph | 83 | 33 |
| | 16 + 16 ppm | | |
| | 1:1 | | |
| 57 | A-8 + dimethomorph | 95 | 33 |
| | 16 + 63 ppm | | |
| | 1:4 | | |
| 58 | A-27 + dimethomorph | 88 | 50 |
| | 63 + 16 ppm | | |
| | 4:1 | | |
| 59 | A-8 + spiroxamine | 92 | 67 |
| | 63 + 63 ppm | | |
| | 1:1 | | |
| 60 | A-8 + spiroxamine | 67 | 33 |
| | 16 + 63 ppm | | |
| | 1:4 | | |
| 61 | A-27 + spiroxamine | 92 | 50 |
| | 63 + 16 ppm | | |
| | 4:1 | | |
| 62 | A-8 + prochloraz | 67 | 33 |
| | 16 + 4 ppm | | |
| | 4:1 | | |
| 63 | A-27 + prochloraz | 83 | 17 |
| | 4 + 16 ppm | | |
| | 1:4 | | |
| 64 | A-27 + pyrimethanil | 92 | 50 |
| | 63 + 16 ppm | | |
| | 4:1 | | |
| 65 | A-27 + pyrimethanil | 67 | 33 |
| | 4 + 16 ppm | | |
| | 1:4 | | |

*)efficacy calculated using Colby's formula

### Use Example 7

### Activity Against Net Blotch of Barley Caused by Pyrenophora Teres, 1 Day Protective Application

In a further test according to use example 4, the following results were obtained:

TABLE 5
Individual active compounds

| | | Active compound | Efficacy in % |
|---|---|---|---|
| | | concentration in the | of the |
| Example | Active compound | spray liquor [ppm] | untreated control |
| 66 | Control (untreated) | — | (90% infection) |
| 67 | A-8 | 63 | 44 |
| 68 | A-31 | 63 | 67 |
| | | 16 | 33 |
| 69 | spiroxamine | 63 | 67 |
| | | 16 | 33 |
| 70 | dithianon | 16 | 0 |

TABLE 6
Mixtures according to the invention

| | Active compound mixture | | |
|---|---|---|---|
| | Concentration | | Calculated |
| Example | Mixing ratio | Observed efficacy | efficacy*) |
| 71 | A-31 + spiroxamine | 83 | 67 |
| | 63 + 16 ppm | | |
| | 4:1 | | |
| 72 | A-31 + spiroxamine | 67 | 33 |
| | 16 + 63 ppm | | |
| | 1:4 | | |
| 73 | A-8 + dithianon | 56 | 44 |
| | 63 + 16 ppm | | |
| | 4:1 | | |

*)efficacy calculated using Colby's formula

### Use example 8

### Activity Against Mildew of Wheat Caused by Erysiphe [Syn. Blumeria] Graminis Forma Specialis. Tritici

The leaves of potted wheat seedlings were sprayed to run off point with an aqueous suspension having the active compound concentration stated below. The suspension or emulsion had been prepared as described above. 24 hours after the spray coating had dried on, the plants were dusted with spores of mildew of wheat (Erysiphe [syn. Blumeria] graminis) form a specialis. tritici). The test plants were then placed in the greenhouse at temperatures between 20 and 24° C. and 60 to 90% relative atmospheric humidity. After 7 days, the extent of the mildew development was determined visually in % infection of the entire leaf area.

1. A fungicidal mixture for controlling harmful fungi, which mixture comprises
1) a 5-chloro-6-phenyl-7-heterocyclylaminotriazolopyrimidine of the formula I, 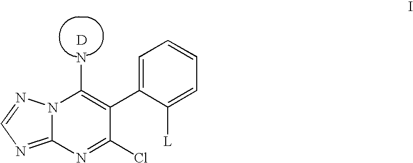
in which
D together with the nitrogen atom forms a pyrrolidine, piperidine or azepine ring, which rings are unsubstituted or substituted by one or two methyl groups or by an ethyl, propyl or butyl group; and
L is methyl, fluorine or chlorine;
and
2) at least one active compound II selected from the following groups:
A) azoles, such as bitertanol, bromoconazole, cyproconazole, difenoconazole, dinitroconazole, epoxiconazole, fenbuconazole, fluquinconazole, flusilazole, flutriafol, hexaconazole, imazalil, ipconazole, metconazole, myclobutanil, penconazole, propiconazole, prochloraz, prothioconazole, simeconazole, tebuconazole, tetraconazole, triadimefon, triadimenol, triflumizole, triticonazole;
B) strobilurins, such as azoxystrobin, dimoxystrobin, fluoxastrobin, kresoxim-methyl, metominostrobin, orysastrobin, picoxystrobin, pyraclostrobin, trifloxystrobin or methyl 2-(ortho-(2,5-dimethylphenyloxy-methylene)phenyl)-3-methoxyacrylate;
C) acylalanines, such as benalaxyl, metalaxyl, mefenoxam, ofurace, oxadixyl;
D) amine derivatives, such as aldimorph, dodine, dodemorph, fenpropimorph, fenpropidin, guazatine, iminoctadine, spiroxamine, tridemorph;
E) anilinopyrimidines, such as pyrimethanil, mepanipyrim or cyprodinil;
F) dicarboximides, such as iprodione, myclozolin, procymidone, vinclozolin;
G) cinnamides and analogs, such as dimethomorph, flumetover or flumorph;
H) antibiotics, such as cycloheximide, griseofulvin, kasugamycin, natamycin, polyoxin or streptomycin;
K) dithiocarbamates, such as ferbam, nabam, maneb, mancozeb, metam, metiram, propineb, polycarbamate, thiram, ziram, zineb;
L) heterocyclic compounds, selected from anilazine, benomyl, boscalid, carbendazim, carboxin, oxycarboxin, cyazofamid, dazomet, dithianon, famoxadone, fenamidone, fenarimol, fuberidazole, flutolanil, furametpyr, iso-prothiolan, mepronil, nuarimol, picobenzamid, probenazole, proquinazid, pyrifenox, pyroquilon, quinoxyfen, silthiofam, thiabendazole, thifluzamide, thiophanate-methyl, tiadinil, tricyclazole, triforine, 3-[5-(4-chloro-phenyl)-2,3-dimethylisoxazolidin-3-yl]pyridine, methyl (2-chloro-5-[1-(3-methylbenzyloxyimino)ethyl]benzyl)carbamate, methyl (2-chloro-5-[1-(6-methylpyridin-2-yl-methoxyimino)ethyl]benzyl)carbamate, benzimidazole derivatives of the formula IIA 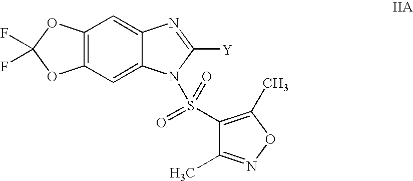
in which Y is chlorine or bromine;
sulfamoyl compounds of the formula III 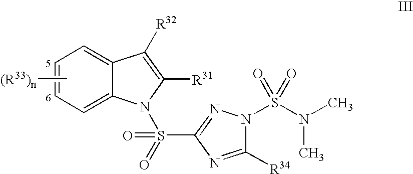
in which the substituents are as defined below:
R³¹ is hydrogen, halogen, cyano, C₁-C₄-alkyl, C₁-C₂-haloalkyl, C₁-C₄-alkoxy, C₁-C₄-alkylthio, C₁-C₄-alkoxycarbonyl, phenyl, benzyl, fommyl or CH═NoR³¹¹;
R³¹¹ is hydrogen, C₁-C₄-alkyl, C₁-C₄-alkylcarbonyl;
R³² is hydrogen, halogen, cyano, C₁-C₄-alkyl, C₁-C₂-haloalkyl, C₁-C₆-alkoxy-carbonyl;
R³³ is halogen, cyano, nitro, C₁-C₄-alkyl, C₁-C₂-haloalkyl, C₁-C₄-alkoxy, C₁-C₄-alkylthio, C₁-C₄-alkoxycarbonyl, formyl or CH═NOR³¹¹;
n is 0, 1, 2, 3 or 4;
R³⁴ is hydrogen, halogen, cyano, C₁-C₄-alkyl or C₁-C₂-haloalkyl;
or
thiophene derivatives of the formula IV 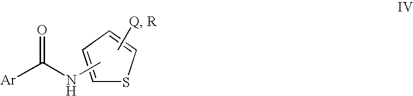
in which the variables are as defined below:
Ar is phenyl or a five- or six-membered aromatic heterocycle which contains one to four heteroatoms from the group consisting of O, N and S, where the cycles are unsubstituted or may be substituted by one to three groups R⁴¹
R⁴¹ is halogen, C₁-C₄-alkyl or C₁-C₄-haloalkyl;
R is phenyl, C₁-C₈-alkyl, C₁-C₈-haloalkyl, C₁-C₈-alkoxy, C₁-C₈-haloalkoxy;
Q is hydrogen, C₁-C₈-alkyl, C₁-C₈-haloalkyl, C₁-C₈-alkoxy, C₁-C₈-haloalkoxy;
M) sulfur and copper fungicides, such as Bordeaux mixtures, copper acetate, copper oxychloride, basic copper sulfate;
N) nitrophenyl derivatives, such as binapacryl, dinocap, dinobuton, nitrophthal-isopropyl;
(O) phenylpyrroles, such as fenpiclonil or fludioxonil;
P) sulfenic acid derivatives, such as captafol, captan, dichlofluanid, folpet, tolylfluanid;
Q) other fungicides, such as acibenzolar-S-methyl, benthiavalicarb, carpropamid, chlorothalonil, cyflufenamid, cymoxanil, dazomet, diclomezin, diclocymet, diclofluanid, diethofencarb, edifenphos, ethaboxam, fenhexamid, fentin-acetate, fenoxanil, ferimzone, fluazinam, fosetyl, fosetyl-aluminum, phosphorous acid, iprovalicarb, hexachlorobenzene, mandipropamide, metrafenone, pencycuron, propamocarb, phthalide, toloclofos-methyl, quintozene, zoxamide,
oxime ether derivatives of the formula V 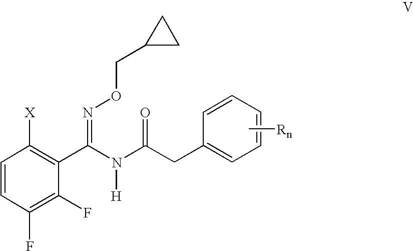
in which
X is C₁-C₄-haloalkoxy,
n is 0, 1, 2 or 3,
R is halogen, C₁-C₄-alkyl, C₁-C₄-haloalkyl, C₁-C₄-alkoxy or haloalkoxy; phenylamidine derivatives of the formula VI 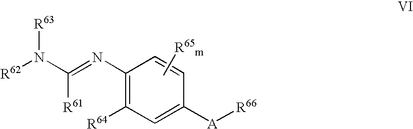
in which the variables are as defined below:
R⁶¹ is hydrogen, C₁-C₈-alkyl, C₂-C₈-alkenyl or C₂-C₈-alkynyl, which are unsubstituted or may be substituted by one to three groups R^{a}:
R^{a} is halogen, C₁-C₈-alkoxy, C₁-C₈-haloalkoxy, C₁-C₈-alkylthio or phenyl which may be substituted by halogen, C₁-C₈-alkyl, C₁-C₈-haloalkyl, C₁-C₈-alkoxy, C₁-C₈-haloalkoxy or C₁-C₈-alkylthio;
R⁶²,R⁶³ can be identical or different and are hydrogen, cyano, C₁-C₈-alkyl, C₂-C₈-alkenyl, C₂-C₈-alkynyl, C₁-C₈-alkoxy, C₁-C₈-alkoxyalkyl, benzyloxy or C₁-C₈-alkylcarbonyl, which are unsubstituted or may be substituted by one to three groups R^{a};
R⁶⁴ is hydrogen, C₁-C₈-alkyl, C₂-C₈-alkenyl or C₂-C₈-alkynyl, which are unsubstituted or may be substituted by one to three groups R^{b}:
R^{b} is one of the groups mentioned under R^{a}, cyano, C(═O)R^{c}, C(═S)R^{c} or S(O)ₚR^{c},
R^{c} is C₁-C₈-alkyl, C₁-C₈-haloalkyl, C₁-C₈-alkoxy, C₁-C₈-haloalkoxy, C₁-C₈-alkylthio, amino, C₁-C₈-alkylamino, di(C₁-C₈-alkyl)amino or phenyl which may be substituted by halogen, C₁-C₈-alkyl, C₁-C₈-haloalkyl, C₁-C₈-alkoxy, C₁-C₈-haloalkoxy or C₁-C₈-alkylthio;
m is 0 or 1;
R⁶⁵ is one of the groups mentioned under R⁶⁴;
A is a direct bond, —O—, —S—, NR^{d}, CHR^{e} or —O—CHR^{e}; R^{d},R^{e} are one of the groups mentioned under R^{a};
R⁶⁶ is phenyl or a five- or six-membered saturated, partially unsaturated or aromatic heterocycle which contains one to four heteroatoms from the group consisting of O, N or S, where the groups R⁶⁶ are unsubstituted or may be substituted by one to three R^{f}:
R^{f} is one of the groups mentioned under R^{b} or amino, C₁-C₈-alkylamino, di(C₁-C₈-alkyl)amino, C₁-C₈-haloalkyl, C₁-C₈-alkoxyalkyl, C₂-C₈-alkenyloxyalkyl, C₂-C₈-alkynyloxyalkyl, C₁-C₈-alkylcarbonyloxy-C₁-C₈-alkyl, cyanooxy-C₁-C₈-alkyl, C₃-C₆-cycloalkyl or phenoxy, where the cyclic groups may be substituted by halogen, C₁-C₈-alkyl, C₁-C₈-haloalkyl, C₁-C₈-alkoxy, C₁-C₈-haloalkoxy or C₁-C₈-alkylthio;
or
a compound of the formula VII 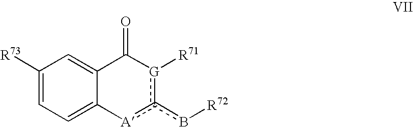
in which the variables are as defined below:
A′ is O or N;
G is C or N;
B is N or a direct bond;
R⁷¹ is C₁-C₄-alkyl;
R⁷² is C₁-C₄-alkoxy; and
R⁷³ is halogen; or
R) growth retardants, such as prohexadione and its salts, trinexapac-ethyl, chlormequat, mepiquat-chloride and diflufenzopyr;
in a synergistically effective amount.

2. The fungicidal mixture according to claim 1, comprising a compound of the formula I and an active compound II in a weight ratio of from 100:1 to 1:100.

3. A compound of the formula I according to claim 1 in which L is methyl and D has one of the meanings below:
—(CH₂)₄—; —CH₂—CH(CH₃)—CH₂—CH₂—; —CH(CH₃)—CH(CH₃)—CH₂—CH₂—; —CH₂—CH(CH₃)—CH(CH₃)—CH₂—; —CH(CH₃)—CH₂—CH(CH₃)—CH₂—; —(CH₂)₅—; —CH(CH₃)—CH₂—CH₂—CH₂—CH₂—; —CH(CH₃)—CH₂—CH₂—CH(CH₃)—; —CH₂—CH₂—CH(CH₃)—CH₂—CH₂—; —CH(CH₃)—CH(CH₃)—CH₂—CH₂—CH₂—; —CH(CH₃)—CH₂—CH(CH₃)—CH₂—CH₂—; —CH(CH₃)—CH₂—CH₂—CH(CH₃)—CH₂—; —CH(CH₃)—CH₂—CH₂—CH₂—CH(CH₃)—; —CH₂—CH(CH₃)—CH(CH₃)—(CH₂)₂—; —CH₂—CH(CH₃)—CH₂—CH(CH₃)—CH₂—; —(CH₂)₂—CH(CH[CH₃]₂)—(CH₂)₂—; —CH(CH₂CH₃)—CH₂—CH₂—CH₂—CH₂—; —CH—CH(CH₂CH₃)—CH₂—CH₂—CH₂—; —CH₂—CH₂—CH(CH₂CH₃)—CH₂—CH₂—; —CH₂—CH₂—CH(CH₂CH₂CH₃)—CH₂—CH₂—; —CH₂—CH₂—CH(C[CH₃]₃)—CH₂—CH₂— and —(CH₂)₆—.

4. A compound of the formula I according to claim 1 in which L is fluorine and D has one of the meanings below:
—(CH₂)₄—; —CH₂—CH(CH₃)—CH₂—CH₂—; —CH(CH₃)—CH(CH₃)—CH₂—CH₂—; —CH(CH₃)—CH₂—CH(CH₃)—CH₂—; —CH(CH₃)—CH₂—CH₂—CH(CH₃)—; —CH₂—CH(CH₃)—CH₂—CH₂—CH₂—; —CH₂—CH₂—CH(CH₃)—CH₂—CH₂—; —CH₂—CH(CH₃)—CH(CH₃)—CH₂—; —CH(CH₃)—CH(CH₃)—CH₂—CH₂—CH₂—; —CH(CH₃)—CH₂—CH(CH₃)—CH₂—CH₂—; —CH(CH₃)—CH₂—CH₂—CH(CH₃)—CH₂—; —CH(CH₃)—CH₂—CH₂—CH₂—CH(CH₃)—; —CH₂—CH(CH₃)—CH(CH₃)—(CH₂)₂—; —CH₂—CH(CH₃)—CH₂—CH(CH₃)—CH₂—; —(CH₂)₂—CH(CH[CH₃]₂)—(CH₂)₂—; —CH(CH₂CH₃)—CH₂—CH₂—CH₂—CH₂—; —CH—CH(CH₂CH₃)—CH₂—CH₂—CH₂—; —CH₂—CH₂—CH(CH₂CH₃)—CH₂—CH₂—; —CH₂—CH₂—CH(CH₂CH₂CH₃)—CH₂—CH₂— and —CH₂—CH₂—CH(C[CH₃]₃)—CH₂—CH₂—.

5. A compound of the formula I according to claim 1 in which L is chlorine and D has one of the meanings below:
—CH(CH₃)—CH(CH₃)—CH₂—CH₂—; —CH₂—CH(CH₃)—CH₂—CH₂—; —CH(CH₃)—CH₂—CH(CH₃)—CH₂—; —CH(CH₃)—CH₂—CH₂—CH(CH₃)—; —CH₂—CH(CH₃)—CH(CH₃)—CH₂—; —CH(CH₃)—CH(CH₃)—CH₂—CH₂—CH₂—; —CH(CH₃)—CH₂—CH₂—CH(CH₃)—CH₂—; —CH(CH₃)—CH₂—CH₂—CH₂—CH(CH₃)—; —CH₂—CH(CH₃)—CH(CH₃)—(CH₂)₂—; —(CH₂)₂—CH(CH[CH₃]₂)—(CH₂)₂—; —CH(CH₂CH₃)—CH₂—CH₂—CH₂—CH₂—; —CH—CH(CH₂CH₃)—CH₂—CH₂—CH₂—; —CH₂—CH₂—CH(CH₂CH₃)—CH₂—CH₂—; —CH₂—CH₂—CH(CH₂CH₂CH₃)—CH₂—CH₂— and —CH₂—CH₂—CH(C[CH₃]₃)—CH₂—CH₂—.

6. A process for preparing the compounds of the formula I according to claim 3 by reacting a dichlorotriazolopyrimidine of the formula II′ 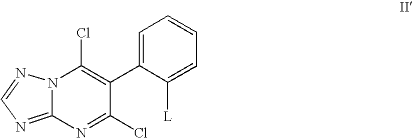
with amines of the formula III′, 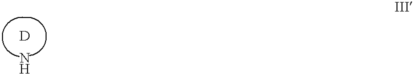
to give compounds of the formula I.

7. A composition comprising a liquid or solid carrier and a mixture according to claim 1 or a compound of the formula I.

8. A method for controlling phytopathogenic harmful fungi which comprises treating the fungi, their habitat or the seed, the soil or the plants to be protected against fungal attack with an effective amount of a compound I and of an active compound from groups A) to R) according to claim 1 or a compound of the formula I.

9. The method according to claim 8, wherein the compound I and the active compound II from groups A) to R) according to claim 1 are applied simultaneously, that is jointly or separately, or in succession.

10. The method according to claim 8, wherein the compound I and the active compound II from groups A) to R) or the mixtures are applied in an amount of from 5 g/ha to 2000 g/ha.

11. The method according to claim 8, wherein the compound I and the active compound II from groups A) to R) or the mixture are/is applied in an amount of from 1 to 1000 g/100 kg of seed.

12. Seed comprising the mixture according to claim 1 or a compound I in an amount of from 1 to 1000 g/100 kg.

13. The use of the compounds I and of the active compounds from groups A) to R) according to claim 1 for preparing a composition suitable for controlling harmful fungi.

14. A process for preparing the compounds of the formula I according to claim 4 by reacting a dichlorotriazolopyrimidine of the formula II′ 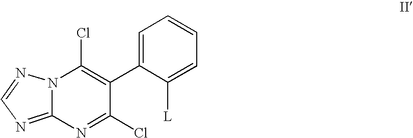
with amines of the formula III′, 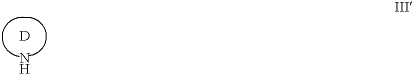
to give compounds of the formula I.

15. A process for preparing the compounds of the formula I according to claim 5 by reacting a dichlorotriazolopyrimidine of the formula II′ 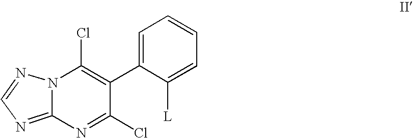
with amines of the formula III′, 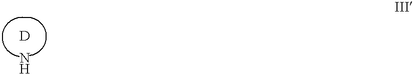
to give compounds of the formula I.

16. A composition comprising a liquid or solid carrier and a mixture according to claim 2 or a compound of the formula I.

17. A composition comprising a liquid or solid carrier and a mixture or a compound of the formula I according to claim 3.

18. A composition comprising a liquid or solid carrier and a mixture or a compound of the formula I according to claim 4.

19. A composition comprising a liquid or solid carrier and a mixture or a compound of the formula I according to claim 5.

20. A method for controlling phytopathogenic harmful fungi which comprises treating the fungi, their habitat or the seed, the soil or the plants to be protected against fungal attack with an effective amount of a compound I and of an active compound from groups A) to R) or a compound of the formula I according to claim 3.
